(19) United States
(12) Patent Application Publication
(10) Pub. No.: US 2018/0080091 A1
(43) Pub. Date: Mar. 22, 2018

(54) DETECTION OF INFLUENZA B VIRUSES

(71) Applicant: THE UNITED STATES OF AMERICA, AS REPRESENTED BY THE SECRETARY, DEPARTMENT OF HEALTH AND HUMAN, Rockville, MD (US)

(72) Inventors: Shannon Lynn Emery, Atlanta, GA (US); Stephen Lindstrom, Chamblee, GA (US); Kai-Hui Wu, Johns Creek, GA (US); LaShondra Shealey Berman, Decatur, GA (US); Christine Marie Warnes, Alpharetta, GA (US); Bo Shu, Lilburn, GA (US)

(73) Assignee: THE UNITED STATES OF AMERICA, AS REPRESENTED BY THE SECRETARY, DEPARTMENT OF HEALTH AND HUMAN, Rockville, MD (US)

(21) Appl. No.: 15/712,861

(22) Filed: Sep. 22, 2017

Related U.S. Application Data
(60) Provisional application No. 62/398,204, filed on Sep. 22, 2016.

(51) Int. Cl. C12Q 1/70

(52) U.S. Cl. CPC C12Q 1/701; G01N 2800/26

(57) ABSTRACT

Primers, probes and kits for detection and lineage differentiation of influenza B virus strains are provided. Also provided are the corresponding assays and methods.

### PRIOR RELATED APPLICATIONS

This application claims the benefit of U.S. Provisional Application No. 62/398,204, filed Sep. 22, 2016, which is incorporated herein by reference in its entirety.

### FIELD

The invention is related to compositions and method for the detection of influenza B viruses.

### BACKGROUND

Influenza B (InfB) viruses cause seasonal respiratory infections throughout the world. Since the 1980's, InfB viruses have evolved into two distinct, co-circulating antigenic lineages, B/Yamagata/16/88 (YAM) and B/Victoria/2/87 (VIC) which are genetically distinguishable. For both clinical and epidemiological reasons, it is important to have sensitive and specific diagnostics tests that can distinguish between the lineages of InfB viruses. For example, current World Health Organization (WHO) recommendations for the formulation of trivalent influenza vaccines include a representative from only one of the two lineages of InfB viruses. Therefore, timely and accurate surveillance information is crucial in order to make reliable seasonal vaccine recommendations.

### SUMMARY

As described below, the inventors have discovered PCR primers and probes that are useful for PCR-based detection and differentiation of InfB Yamagata and Victoria strains with high specificity and sensitivity, as well as superior LOD. The primer and the probes discovered by the inventors are based on the sequences of HA1 domain of hemagglutinin (HA) segment of the viral genome and can be used in the detection methods that employ reverse transcriptase polymerase chain reaction (RT-PCR) techniques that monitor the amplification of InfB virus genetic RNA in real time (rRT-PCR). Among other things, the inventors developed an rRT-PCR assay for detection of a lineage of InfB virus using HA gene segment sequences. The assay, which can be referred to in this letter as “InfB lineage assay,” specifically detects InfB viruses of Yamagata and Victoria lineages, and uses the primers and the probes described further in this document. The primers and the probes discovered by the inventors can be combined in kits for conducting such assays. Accordingly, the present invention provides PCR primers, PCR probes, methods of using the PCR primers and/or probes, as well as the kits comprising the probes and/or primers. Embodiments of the present invention can be used in clinical, research and public health fields. For example, embodiments of the present invention can be used to determine if samples of interest, such as those obtained from humans or animals, contain an InfB virus strain of Victoria or Yamagata lineage.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are further described in the Detailed Description section below.

Some of the embodiments of the present invention are summarized below. This summary is not intended to identify key or essential features of the claimed subject matter, nor is it intended to be used in isolation to determine the scope of the claimed subject matter. The subject matter should be understood by reference to appropriate portions of the entire specification and each claim.

Some embodiments of the present invention are probes, such as a probe comprising an oligonucleotide comprising a sequence at least 90% identical to SEQ ID NO:3, SEQ ID NO:6 or SEQ ID NO:8 linked to at least one of a fluorophore moiety and a quencher moiety. The probe can have length of 20 bases or less. In some examples of the probes, the sequence is SEQ ID NO:3, SEQ ID NO:6 or SEQ ID NO:8. In some other examples of the probes, the oligonucleotide consists of SEQ ID NO:3, SEQ ID NO:6 or SEQ ID NO:8. For example, the probe can be an oligonucleotide consisting of SEQ ID NO:3, SEQ ID NO:6 or SEQ ID NO:8 linked to the fluorophore moiety and the quencher moiety. In the probes according to the embodiments of the present invention, the fluorophore moiety can comprise a fluorescein moiety. The fluorophore moiety can be coupled to a 5′ terminus of the probe. The quencher moiety can be a BHQ quencher. The quencher moiety can be coupled to a 3′ terminus of the probe or to an internal base.

Some embodiments of the present invention are primers, such as primer comprising an oligonucleotide having a sequence at least 90% identical to a sequence selected from the group consisting of SEQ ID NO:1, SEQ ID NO:2, SEQ ID NO:4, SEQ ID NO:5, SEQ ID NO:7 and SEQ ID NO:9. In some examples, the primer comprises a detectable label. The primer can have a length of 30 bases or less. In some examples of the primers, the sequence is selected from the group consisting of SEQ ID NO:1, SEQ ID NO:2, SEQ ID NO:4, SEQ ID NO:5, SEQ ID NO:7 and SEQ ID NO:9. In some other examples, the oligonucleotide consists of the sequence selected from the group consisting of SEQ ID NO:1, SEQ ID NO:2, SEQ ID NO:4, SEQ ID NO:5, SEQ ID NO:7 and SEQ ID NO:9.

Some embodiments of the present invention are kids, such as a kit for detecting a nucleic acid sequence of a region of hemagglutinin (HA) gene segment of influenza B virus in a sample (the sample can be an ex vivo sample derived from a human or an animal subject, a laboratory sample, a virus isolate sample or a vaccine sample), comprising at least one probe according to the embodiments of the present invention and other reagents for performing a real time reverse transcriptase PCR (rRT-PCR) assay. In some examples of the kit, the other reagents comprise at least one primer comprising a sequence at least 90% identical to a sequence selected from the group consisting of SEQ ID NO:1, SEQ ID NO:2, SEQ ID NO:4, SEQ ID NO:5, SEQ ID NO:7 and SEQ ID NO:9. An example of a kit comprises a probe comprising the sequence at least 90% identical to SEQ ID NO:3 and at least one primer selected from the group consisting of a first primer comprising a sequence at least 90% identical to SEQ ID NO:1 and a second primer comprising a sequence at least 90% identical to SEQ ID NO:2. Another example of a kit comprises a probe comprising the sequence at least 90% identical to SEQ ID NO:8 and at least one primer selected from the group consisting of a first primer comprising a sequence at least 90% identical to SEQ ID NO:7 and a second primer comprising a sequence at least 90% identical to SEQ ID NO:2. Another example of a kit comprises a probe comprising the sequence at least 90% identical to SEQ ID NO:3 and at least one primer selected from the group consisting of a first primer comprising a sequence at least 90% identical to SEQ ID NO:7 and a second primer comprising a sequence at least 90% identical to SEQ ID NO:2. Another example of a kit comprises a probe comprising the sequence at least 90% identical to SEQ ID NO:3 and at least one primer selected from the group consisting of a first primer comprising a sequence at least 90% identical to SEQ ID NO:7 and a second primer comprising a sequence at least 90% identical to SEQ ID NO:2. Yet another example of a kit comprises a probe comprising the sequence at least 90% identical to SEQ ID NO:6 and at least one primer selected from the group consisting of a first primer comprising a sequence at least 90% identical to a sequence SEQ ID NO:4 and a second primer comprising a sequence at least 90% identical to a sequence SEQ ID NO:5. Yet another example of a kit comprises a probe comprising the sequence at least 90% identical to SEQ ID NO:6 and at least one primer selected from the group consisting of a first primer comprising a sequence at least 90% identical to a sequence SEQ ID NO:4 and a second primer comprising a sequence at least 90% identical to a sequence SEQ ID NO:9.

One example of a kit is a kit for amplifying a nucleic acid sequence of a region of hemagglutinin (HA) gene segment of influenza B virus in a sample, comprising at least one primer according to the embodiments of the present invention and one or more other ingredients for performing a PCR. Yet one more example is a kit for amplifying a region of hemagglutinin (HA) gene segment of influenza B virus in a sample, comprising at least one of: one or both first and second primers for amplifying a region of HA gene of Victoria lineage InfB virus strain, wherein the first primer is an oligonucleotide of SEQ ID NO:1 or SEQ ID NO:7, an oligonucleotide comprising SEQ ID NO:1 or SEQ ID NO:7, an oligonucleotide of a sequence at least 90% identical to SEQ ID NO:1 or SEQ ID NO:7, or an oligonucleotide comprising a sequence at least 90% identical to SEQ ID NO:1 or SEQ ID NO:7; wherein the second primer is an oligonucleotide of SEQ ID NO:2, an oligonucleotide comprising SEQ ID NO:2, an oligonucleotide of a sequence at least 90% identical to SEQ ID NO:2, or an oligonucleotide of a sequence at least 90% identical to SEQ ID NO:2. Yet one more example is a kit for amplifying a region of hemagglutinin (HA) gene segment of influenza B virus in a sample, comprising at least one or both third and fourth primers for amplifying a region of HA gene of Yamagata lineage InfB virus strain, wherein the third primer is an oligonucleotide of SEQ ID NO:4, an oligonucleotide comprising SEQ ID NO:4, an oligonucleotide of a sequence at least 90% identical to SEQ ID NO:4, or an oligonucleotide comprising a sequence at least 90% identical to SEQ ID NO:4; wherein the fourth primer is an oligonucleotide of SEQ ID NO:5 or SEQ ID NO:9, an oligonucleotide comprising SEQ ID NO:5 or SEQ ID NO:9, an oligonucleotide of a sequence at least 90% identical to SEQ ID NO:5 or SEQ ID NO:9, or an oligonucleotide comprising a sequence at least 90% identical to SEQ ID NO:5 or SEQ ID NO:9. The kits according to the embodiments of the present invention can comprise both the primers for amplifying a region of HA gene of Victoria lineage InfB virus strain and the primers for amplifying a region of HA gene of Yamagata lineage InfB virus, in various combinations. The above kits can comprise one or more other reagents for performing a PCR. The one or more other reagents for the kit can be the reagents for performing RT-PCR, such as the reagents for performing rRT-PCR. In the kits according to the embodiments of the present invention, at least one of the primers can comprise a detectable moiety. In the above kits according to the embodiments of the present invention, the other reagents can comprise one or more probes or one or more additional primers. For example, the other reagents can comprise at least one of a first probe, the first probe comprising an oligonucleotide comprising a sequence at least 90% identical to SEQ ID NO:3 or SEQ ID NO:8 (such as SEQ ID NO:3 or SEQ ID NO:8), if the one or both primers for amplifying a region of HA gene of Victoria lineage InfB virus strain are present in the kit, and at least one of a second probe comprising an oligonucleotide comprising a sequence at least 90% identical to SEQ ID NO:6 (such as SEQ ID NO:6), if one or both primers for amplifying a region of HA gene of Yamagata lineage InfB virus strain are present in the kit.

Some other examples of the kits according to the embodiments of the present invention are kits comprising probes for detection of an amplified region of HA gene of Victoria lineage InfB virus strain and/or amplified region of HA gene of Yamagata lineage InfB virus strain. Such kits can comprise at least one of a first probe, at least one of a second probe, or both the at least one of the first probe and the at least one of the second probe. In the context of the kits, the first probe or probes is for detection of an amplified region of HA gene of Victoria lineage InfB virus strain, and the second probe or probes is for detection of an amplified region of HA gene of Yamagata lineage InfB virus strain. An example of a first probe is an oligonucleotide comprising a sequence at least 90% identical to SEQ ID NO:3 or SEQ ID NO:8 (such as SEQ ID NO:3 or SEQ ID NO:8). An example of a second probe is an oligonucleotide comprising a sequence at least 90% identical to SEQ ID NO:6 (such as SEQ ID NO:6). The one or more first probe and the one or more second probe can each comprise a fluorophore moiety and a quencher moiety. The fluorophore moieties of the first and the second probe can be same or different, and wherein the quencher moieties of the first and the second probe can be same or different. The above kits can comprise one or more other reagents for performing a PCR. The one or more other reagents for the kit can be the reagents for performing RT-PCR, such as the reagents for performing rRT-PCR. The kits according to the embodiments of the present invention, the other reagents can comprise primers described elsewhere in this document, other primers or other probes, in various combinations.

Some embodiments of the present invention are methods, such as methods of detecting a presence or absence of InfB influenza strain in a sample, wherein the influenza virus strain comprises a region of HA gene of Victoria lineage InfB virus. In one example, the method comprises the steps of contacting the sample with reagents for performing rRT-PCR and a probe according to the embodiments of the present invention specific for the region of HA gene of Victoria lineage InfB virus (VIC probe) and forward and reverse primers specific for the region of HA gene of Victoria lineage InfB virus; performing rRT-PCR on the sample to generate a PCR cycle threshold; and, comparing the PCR cycle threshold to a control value, wherein if the cycle threshold is below the control value, the InfB virus strain is absent from the sample, and wherein if the cycle threshold is above the control value, the InfB virus strain is present in the sample. In the above method, the sample can be contacted with a VIC probe and a forward primer comprising a sequence at least 90% identical to SEQ ID NO:1 or SEQ ID NO:7. The sample can be contacted with the VIC probe and a reverse primer comprising a sequence at least 90% identical to SEQ ID NO:2. Another example of a method of detecting a presence or absence of InfB influenza virus strain in a sample, wherein the influenza virus strain comprises a region of HA gene of Yamagata lineage InfB virus, is the method comprising: the steps of contacting the sample with reagents for performing rRT-PCR and a probe according to the embodiments of the present invention specific for the region of HA gene of Yamagata lineage InfB virus (YAM probe) and forward and reverse primers specific for the region of HA gene of Yamagata lineage InfB virus (YAM primers); performing rRT-PCR on the sample to generate a PCR cycle threshold; and, comparing the PCR cycle threshold to a control value, wherein if the cycle threshold is below the control value, the InfB virus strain is absent from the sample, and wherein if the cycle threshold is above the control value, the InfB virus strain is present in the sample. In the above method, the sample can be contacted with the YAM probe and a forward primer comprising a sequence at least 90% identical to SEQ ID NO:4. The sample can be contacted with the YAM probe and a reverse primer comprising a sequence at least 90% identical to SEQ ID NO:5 or SEQ ID NO:9. The above methods can comprise a step of determining a quantity of the InfB virus strain in the sample when the InfB virus strain is present in the sample. In any of the above methods, the steps of comparing, determining the quantity, or both, can be performed by a computer.

The methods according to the embodiments of the present invention also include methods of amplifying a nucleic acid sequence, such as a method comprising the steps of: contacting the sample with at least one primer according to the embodiments of the present invention; and, performing a PCR. In the above method, the sample can be contacted with a first primer comprising the sequence at least 90% identical to SEQ ID NO:1 or SEQ ID NO:7 and a second primer comprising the sequence at least 90% identical to SEQ ID NO:2, or the sample can be contacted with a third primer comprising the sequence at least 90% identical to SEQ ID NO:4 and a fourth primer comprising the sequence at least 90% identical to SEQ ID NO:5 or SEQ ID NO:9. The PCR can be RT-PCR. Also included among the embodiments of the present invention are methods of detecting a nucleic acid comprising a region of HA segment of InfB virus in the sample, comprising the steps of: performing a method of amplifying a nucleic acid sequence according to the embodiments of the present invention; and detecting one or more products of the amplification, wherein the nucleic acid is present in the sample if the one or more products of the amplification are detected. In an example of the above method, PCR is rRT-PCR and the detecting is performed using a probe according to the embodiments of the present invention comprising a sequence at least 90% identical to SEQ ID NO:3 or SEQ ID NO:8 In another example of the above method, the PCR is rRT-PCR and the detecting is performed using a probe according to the embodiments of the present invention comprising a sequence at least 90% identical to SEQ ID NO:6. The above methods can further comprise a step of determining a quantity of the nucleic acid comprising the region of HA segment of InfB virus, then the nucleic acid is present in the sample.

The methods according to the embodiments of present invention can be performed on ex vivo samples derived from a human or an animal subject, laboratory samples, virus isolate samples, or vaccine samples. For example, a method of determining if a subject is infected with an InfB virus strain, wherein the InfB virus strain is of Victoria lineage, comprises the steps of: contacting a sample derived from the subject with reagents for performing rRT-PCR and a probe according to the embodiments of the present invention comprising a sequence at least 90% identical to SEQ ID NO:3 or SEQ ID NO:8 (VIC probe) and other reagents for performing rRT-PCR; performing rRT-PCR on the sample to generate a PCR cycle threshold; and, comparing the PCR cycle threshold to a control value, wherein if the cycle threshold is below the control value, the subject is not infected with the a Yamagata lineage InfB virus strain, and wherein if the cycle threshold is above the control value, the subject is infected with the Yamagata lineage InfB virus strain. In the above method, the subject can be contacted with the VIC probe, a first primer comprising a sequence at least 90% identical to SEQ ID NO:1 or SEQ ID NO:7, and a second primer comprising a sequence at least 90% identical to SEQ ID NO:2. In another example, a method of determining if a subject is infected with an InfB virus strain, wherein the InfB virus strain is of Yamagata lineage, comprises the steps of: contacting a sample derived from the subject with reagents for performing rRT-PCR and a probe according to the embodiments of the present invention comprising a sequence at least 90% identical to SEQ ID NO:6 (YAM probe) and other reagents for performing rRT-PCR; performing rRT-PCR on the sample to generate a PCR cycle threshold; and, comparing the PCR cycle threshold to a control value, wherein if the cycle threshold is below the control value, the subject is not infected with the a Yamagata lineage InfB virus strain, and wherein if the cycle threshold is above the control value, the subject is infected with the Yamagata lineage InfB virus strain. In the above method, the subject can be contacted with the VIC probe, a first primer comprising a sequence at least 90% identical to SEQ ID NO:4, and a second primer comprising a sequence at least 90% identical to SEQ ID NO:5 or SEQ ID NO:9. The above methods can further comprise a step of determining a quantity of the InfB virus strain in the sample when the InfB virus strain is present in the sample. The steps of comparing, determining the quantity, or both, can be performed by a computer.

Other objects and advantages of the invention will be apparent from the following detailed description of embodiments of the invention.

### BRIEF DESCRIPTION OF THE FIGURES

FIGS. 1A, 1B, 1C and 1D show the examples of the chemical structures of Black Hole Quencher® dyes (Biosearch Technologies, Petaluma, Calif.).

FIG. 2 is a schematic illustration of TaqMan probe.

FIG. 3 is a schematic illustration of Zen probe.

FIG. 4 shows chemical structures of pdU-CE Phosphoramidite (5′-Dimethoxytrityl-5-(1-Propynyl)-2′-deoxyUridine,3′-[(2-cyanoethyl)-(N,N-diisopropyl)]-phosphoramidite) and pdC-CE Phosphoramidite (5 ‘-Dimethoxytrityl-N4-diisobutylaminomethylidene-5-(1-Propynyl)-2’-deoxyCytidine,3 [(2-cyanoethyl)-(N,N-diisopropyl)]-phosphoramidite).

FIG. 5 shows the melt-curve analysis of DNA products amplified by RT-PCR using VIC primers evaluated using 10-fold serial dilutions of viral RNA of VIC lineage InfB strain B/Nevada/03/2011 (VIC). Temperature is plotted on X-axis, and temperature is plotted on Y-axis.

FIG. 6 shows the melt-curve analysis of DNA products amplified by RT-PCR using YAM primers evaluated using 10-fold serial dilutions of viral RNA of YAM lineage InfB strain B/Wisconsin/01/2010. Temperature is plotted on X-axis, and temperature is plotted on Y-axis.

FIG. 7 shows the line plots of RNA dilution factor (X-axis) vs. Cₜ values (Y-axis) illustrating the reaction efficiency of YAM primers and probes. The testing was performed using a five-fold serial dilution of YAM InfB strain B/Wisconsin/10/2010 viral RNA in quadruplicate.

FIG. 8 shows the line plots of RNA dilution factor (X-axis) vs. Cₜ values (Y-axis) illustrating the reaction efficiency of VIC primers and probes. The testing was performed using a five-fold serial dilution of VIC strain B/Nevada/03/2011 viral RNA in quadruplicate.

### DESCRIPTION

### Definitions

The following abbreviations may be used, among others, in the present document: PCR—polymerase chain reaction; RT—reverse transcriptase; RT-PCR—reverse transcriptase PCR; rRT-PCR—real-time RT-PCR; RNA—ribonucleic acid; DNA—deoxyribonucleic acid; HA—hemagluttinin; NA—neuramidase; BHQ-Black Hole Quencher, FAM—6 carboxyfluorescein; FRET—fluorescence resonance energy transfer; LOD—limit of detection.

The term “amplification” and the related terms are used to refer to the process or to the result of the process used to increase the number of copies of a nucleic acid molecule. The resulting products can be called “amplification products” or “amplicons.” An example of an amplification technique is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify a number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

The term “assay” and the related terms are used to broadly refer to methods, processes or procedures used for assessing or measuring the presence, absence or amount or the of a target entity (the analyte). The assays according to the embodiments of the present invention are used to assess the presence, absence or amount of InfB virus in a sample.

The terms “assess,” “assessment,” “assessing” and related terms are used in reference to influenza virus and its genes to describe inferring the presence, the absence or the amount of influenza virus strain in a sample based on the detected presence, absence or amount of influenza virus sequences.

The terms “to contact,” “contacting” and the related terms can be used to describe the process or the result of placing chemical compounds in the same reaction environment, such as the same reaction vessel or solution.

The terms “detect,” “detecting,” “detection” and similar terms are used in this document to broadly to refer to a process of discovering or determining the presence or an absence, as well as a degree, quantity, or level, or probability of occurrence of something. The terms necessarily involve a physical transformation of matter, such as nucleic acid amplification by PCR. For example, the term “detecting” when used in the context of influenza virus detection, can denote discovery or determination of the presence, absence, level or quantity, as well as a probability or likelihood of the presence or absence of the influenza virus being detected. It is to be understood that the expressions “detecting the presence or absence,” “detection of the presence or absence” and related expressions, include qualitative, semi-quantitative and quantitative detection. Quantitative detection includes a determination of the level, quantity or amounts of influenza virus in the sample, on which the detection process is performed. Semi-quantitative detection and qualitative detection include inferring the presence or absence of influenza virus in a sample based on a detection parameter being above or below a predetermined value.

The terms “detection limit,” “limit of detection,” abbreviation “LOD” and other related terms can be used in the context of the embodiments of the present invention to refer to the lowest analyte concentration or amount that can be reliably (for example, reproducibly) detected for a given type of sample and/or assay. Limit of detection can be determined by testing serial dilutions of a sample known to contain the analyte and determining the lowest dilution at which detection occurs. The limit of detection of the assays described in this document can be expressed as level of infectivity (for example, 50% tissue culture infective dose/ml (TCID₅₀/ml) or 50% embryo (or egg) infective dose/ml (EID₅₀/ml), expressed as a log scale), concentration, such as RNA copy number/μl or RNA copy number per reaction volume, or amount, such as the number of copies of a particular sequence that can be detected.

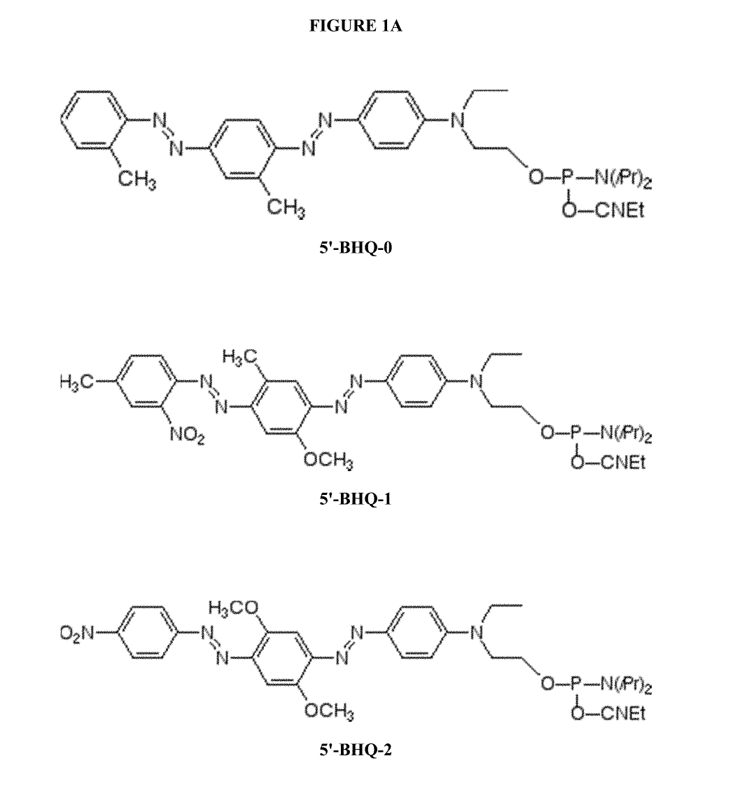

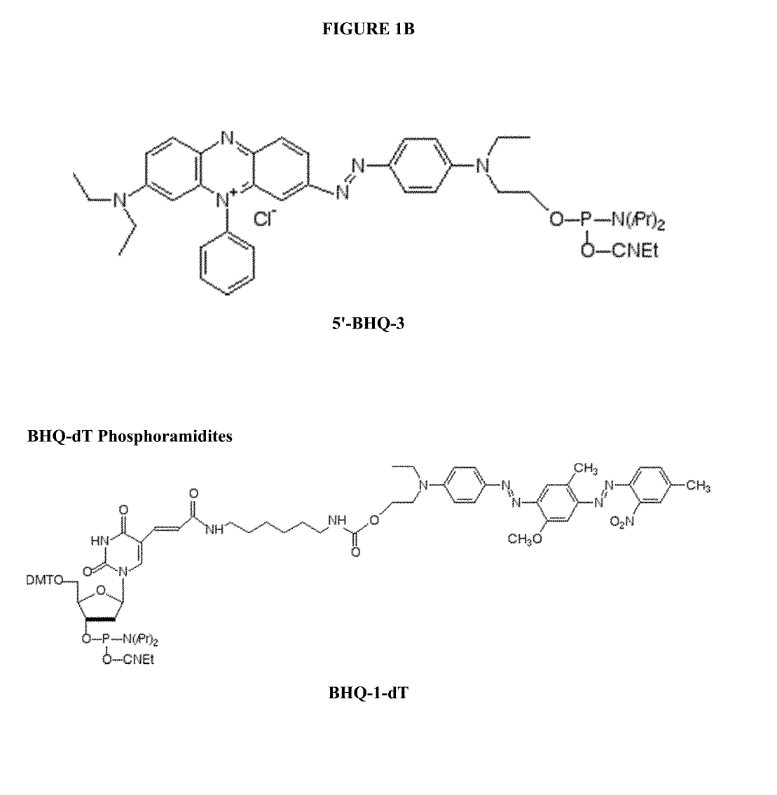

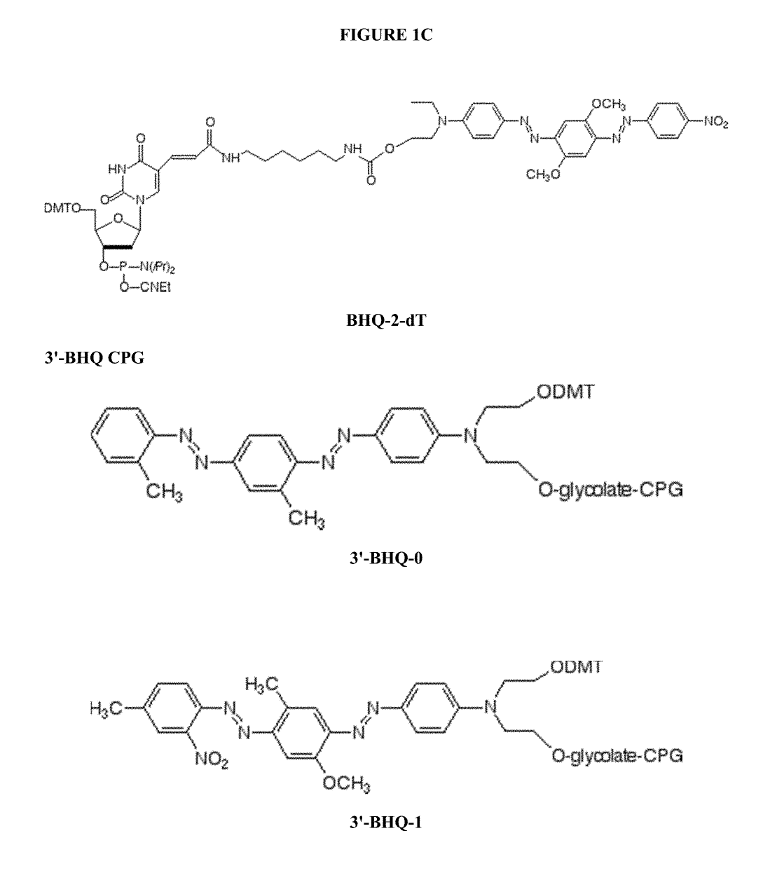

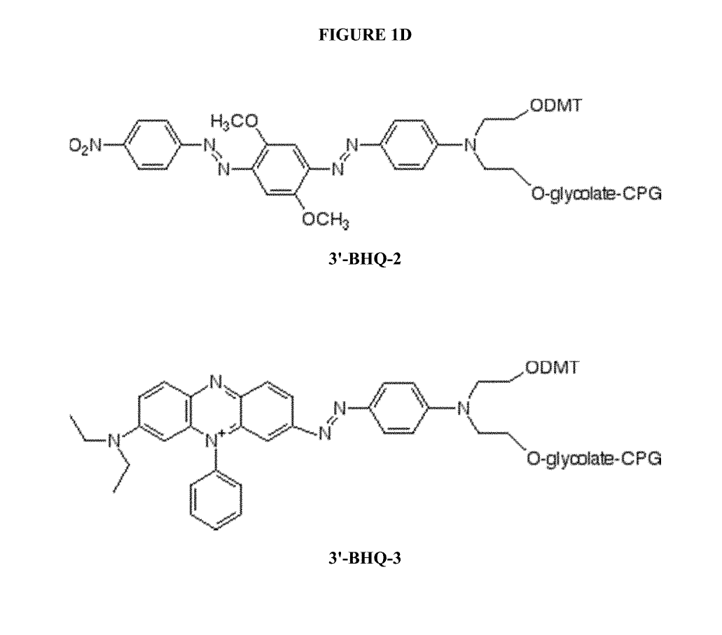

The term “fluorescence” broadly refers to the process or the result of the emission of light by a substance that has absorbed light or other electromagnetic radiation. The following terms and concepts can be used to describe how fluorescence is employed in the embodiments of the present invention. Fluorophores or fluorescent dyes are chemical compounds or moieties that can re-emit light upon light excitation. Fluorophores typically contain several combined aromatic groups, or plane or cyclic molecules with several π bonds. A fluorophore absorbs light energy of a specific wavelength and re-emits light at a longer wavelength. When a fluorophore is excited at a particular wavelength, it is promoted to an excited state. In the absence of a quencher, the excited dye emits light in returning to the ground state. When a quencher is present in physical proximity, the excited fluorophore can return to the ground state by transferring its energy to the quencher, without the emission of light. Different types of quenchers exist. One quenching mechanism relies on the ability of the fluorophore to transfer energy to a second fluorophore by fluorescence resonance energy transfer (FRET). This returns the fluorophore to the ground state and generates the quencher excited state. The quencher then returns to the ground state through emissive decay (fluorescence). In order for this to happen, the emission spectrum of the fluorophore must overlap with the absorption spectrum of the second fluorophore (quencher). One example of such the fluorophore/quencher pair is fluorescein (used as the fluorescent reporter dye) and rhodamine as the quencher (FAM/TAM probes). However, quencher fluorescence can increase background noise due to overlap between the quencher and reporter fluorescence spectra. Dark quenchers are dyes with no native fluorescence. Dark quenchers return from the excited state to the ground state via non-radiative decay pathways, without the emission of light. In dark decay, energy is given off via molecular vibrations (heat). With the typical μM or less concentration of probe, the heat from radiationless decay is too small to affect the temperature of the solution. Thus, the term “dark quencher” can be used in the context of the present invention to refer to a substance or moiety that absorbs excitation energy from a fluorophore and dissipates the energy as heat; while the term “fluorescent quencher” can be used to refer to a substance or moiety that re-emits much of this energy as light. Dark quenchers do not occupy an emission bandwidth and allow multiplexing, when two or more reporter-quencher probes are used together. BHQ quenchers, some of which are illustrated in FIG. 1, are examples of dark quenchers.

Influenza (flu) virus is a member of Orthomyxoviridae family. There are three subtypes of influenza viruses, designated influenza A, influenza B, and influenza C. Human influenza A and B viruses cause seasonal epidemics of disease almost every winter in the United States. The emergence of a novel and different influenza virus strain infecting people can cause an influenza pandemic. Influenza type C infections cause a mild respiratory illness and are not thought to cause epidemics. Influenza virus is an RNA virus and contains a segmented negative-sense RNA genome. That is, influenza type virus genome is not a single piece of RNA; instead, it consists of segmented pieces of negative-sense RNA, which can be referred to as “segments,” each piece containing either one or two genes which code for a gene product (protein). Influenza virus genome encodes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (PA), and nonstructural protein 2 (NS2). A section of the influenza virus RNA encoding a particular protein can be referred to as a “gene,” “segment,” or “gene segment.” The HA, NA, M1 and M2 proteins are membrane associated, whereas NP, PB1, PB2, PA and NS2 are nucleocapsid associated proteins. The HA and NA proteins are envelope glycoproteins, responsible for virus attachment and penetration of the viral particles into the cell, and the sources of the major immunodominant epitopes for virus neutralization and protective immunity. Each influenza virus subtype has mutated into a variety of strains with differing pathogenic profiles. Influenza A viruses are classified into subtypes based on antibody responses to HA and NA. There are 16 H and 9 N subtypes known, but only H 1, 2 and 3, and N 1 and 2 are commonly found in humans. Influenza B (InfB) viruses are not divided into subtypes, but they have evolved into two antigenically and genetically distinct lineages: B/Yamagata and B/Victoria, represented by InfB strains B/Yamagata/16/88 and B/Victoria/2/87. Yamagata and Victoria lineages can be distinguished based on the sequence of HA1 domain of hemagglutinin (HA) segment of the viral genome. This document follows an internationally accepted naming convention for influenza viruses, as published in February 1980 in the Bulletin of the World Health Organization, 58(4):585-591 (1980). This convention uses the following components: the antigenic type (A, B, C); the host of origin (swine, equine, chicken, etc.; for human-origin viruses, no host of origin designation is given)”; geographical origin (Denver, Taiwan, etc.); strain number (15, 7, etc.); year of isolation (57, 2009, etc.). This document uses designations YAM or “Yamagata” to refer to “Yamagata-like” InfB strains or strains of Yamagata lineage, and designations VIC or “Victoria” to refer to “Victoria-like” InfB strains or strains of Victoria lineage. In this document, pathogenic circulating influenza virus strains can be referred to as “circulating strains” or “community-acquired strains.”

The term “isolated” can be used in this document to refer to a biological component (such as a nucleic acid or a virus) that has been substantially separated or purified away from other biological components (such as cell debris, or other proteins or nucleic acids). Biological components that have been “isolated” include those components purified by standard purification methods. The term also embraces recombinant nucleic acids and viruses, as well as chemically synthesized nucleic acids.

“Moiety” refers to a part or functional group of a molecule.

“Oligonucleotide” and related terms are used in this document to refer to nucleic acid molecules, such as RNA or DNA molecules or their modifications, 200 bases long or less. The term “oligonucleotide” includes naturally occurring or non-natural (synthetic) nucleic acid sequences, as well as sequences containing residues, liners, labels etc. that do not naturally occur in nucleic acids, including modified natural nucleotides, etc.

“Primers” (singular—“primer”) are strands of short nucleic acid sequences, such as a DNA oligonucleotides, used as starting points for DNA synthesis during nucleic acid amplification reaction, such as PCR. Primers contain oligonucleotides with a sequences that can be annealed to a complementary target nucleic acid molecule by nucleic acid hybridization to form a hybrid between the primer and the target nucleic acid strand. A primer can be described as “specific” for a target nucleic acid. During the amplification reaction, a primer can be extended along the target nucleic acid molecule by a polymerase enzyme. Thus, primers can be used to amplify a target nucleic acid molecule (such as a portion of an influenza virus nucleic acid), wherein the sequence of the primer is specific for the target nucleic acid molecule, for example so that the primer will hybridize to the target nucleic acid molecule under high or very high stringency hybridization conditions employed in some parts of the PCR cycle. Primers are often characterized by “Primer Melting Temperature” (Tₘ), which is the temperature at which one half of the DNA duplex will dissociate to become single stranded and indicates the duplex stability. Primer melting temperature depends, in part, on its length and nucleotide sequence. A primer according to the embodiments of the present invention can be is at least 15 nucleotides in length, such as at least 15 contiguous nucleotides complementary to a target nucleic acid molecule, including the primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or 50 or more contiguous nucleotides complementary to the target nucleic acid molecule to be amplified, such as a primer of 15-60 nucleotides, 15-50 nucleotides, 20-40 nucleotides, or 15-30 nucleotides. Primers are generally used in pairs for amplification of a nucleic acid sequence, for example, by PCR, real-time PCR, or other nucleic-acid amplification methods. An “upstream” or “forward” primer is a primer 5′ to a reference point on a nucleic acid sequence. A “downstream” or “reverse” primer is a primer 3′ to a reference point on a nucleic acid sequence. At least one forward and one reverse primer are included in an amplification reaction. Primers can contain one or more detectable labels or reporters, meaning moieties that are detectable by various methods or assist in detection. One example of a detectable label or reporters is a fluorescent dye, such as WellRed fluorescent dyes (supplied by Beckman Coulter, Inc.). Another example is biotin. Biotinylated primers can be used, for example, in Luminex technology and Pyrosequencing techniques. Biotin can be added to oligonucleotides on either terminus (“standard” biotin), as well as internally through a modified thymidine residue (biotin-dT). In some cases, primers act as probes during detection. For example, so-called scorpion primers can be used for detection in real-time PCR assays. Scorpion primers contain a stem-and-loop oligonucleotide structure with a 5′ fluorescent report and a 3′ quencher (“probe sequence”), which is attached to 5′ terminus of the oligonucleotide specific for the target nucleic acid sequence. During the annealing phase of the PCR, the probe sequence hybridizes to the newly formed complementary target sequence, separating the fluorophore and the quencher dyes and leading to emission of fluorescence signal.

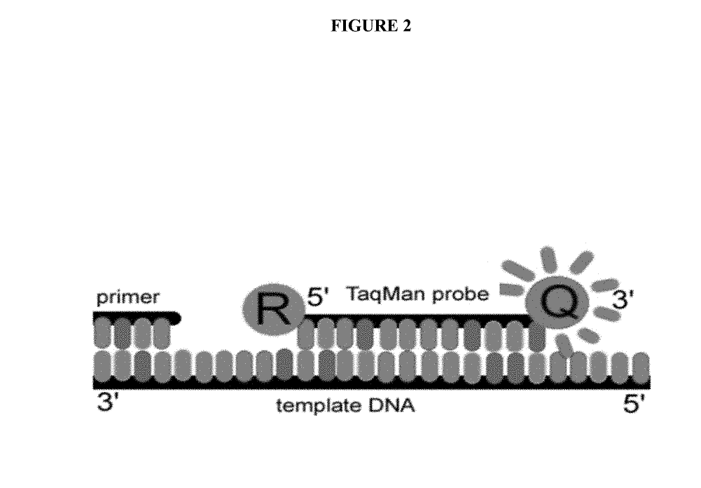

The term “probe” (plural—“probes”) and-related terms are used in this document to refer to a molecule containing an oligonucleotide of variable length that is capable of hybridizing to a target nucleic acid sequence. The probe can be described as “specific for” the target nucleic acid sequence. Probes can be characterized by their Tₘ. The probes according to the embodiments of the present invention include rRT-PCR probes, which are probes capable of hybridizing to rRT-PCR amplification products. A probe can contain one or more detectable labels or reporters, meaning moieties that are detectable by various methods or assist in detection. For example, a variation of the probes described in this document are fluorescent reporter probes useful in rRT-PCR assays. One example of such probes are the so-called hydrolysis probes, such TaqMan® probes. TaqMan® probes are oligonucleotide probes that contain a fluorescence reporter moiety covalently attached to the 5′ end and a quencher moiety, which can be attached at the 3′ end or at an internal nucleotide, which reduces the fluorescence emitted by the fluorescent reporter. FIG. 2 schematically illustrates a TaqMan® probe (R denotes a reporter; Q denotes a quencher). Some examples of fluorophores-suitable for use as fluorescent reporter dyes in TaqMan® probes are 6-carboxyfluorescein (FAM), tetrachlorofluorescein (TET), hexachloro-6-carboxyflourescein (HEX). When a probe is intact, the quencher suppresses the fluorescence of the fluorescence reporter dye. When the probe is used in real-time PCR, during the extension phase, the probe is cleaved by the exonuclease activity of the DNA polymerase, releasing the fluorophore. The fluorophore release results in an increase in fluorescence signal, which is proportionate to the amount of the PCR product.

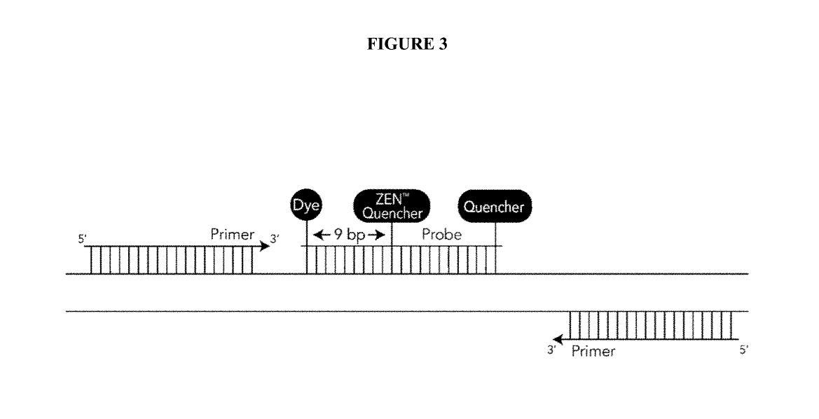

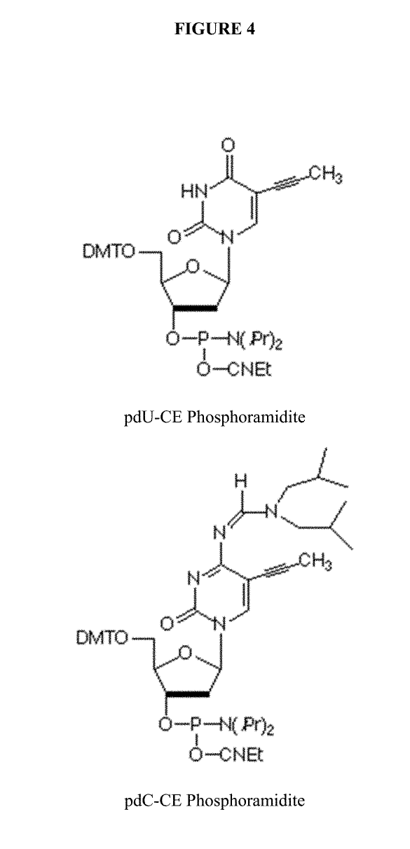

Variations and modifications of hydrolysis probes are possible. One example of such a modification is incorporation-of conjugated Minor Groove Binder (MGB) groups into a probe. The MGB groups act as duplex stabilizers. MGB probes typically incorporate a 5′ reporter dye and a 3′ nonfluorescent quencher, with the MGB moiety attached to the quencher molecule. One example of an MGB moiety is dihydrocyclopyrroloindole tripeptide (DPI₃), which folds into the minor groove formed by the terminal 5-6 bp of the probe. Such probes form extremely stable duplexes with single-stranded DNA targets, allowing shorter probes to be used. In comparison with unmodified DNA, MGB probes have higher melting temperature (Tₘ) and increased specificity. Another example is incorporation of modified bases, such as propyne derivatives, into nucleotides. For example, substitution of C-5 propynyl-dC (pdC) for dC and C-5 propynyl-dU (pdU) for dT (both illustrated in FIG. 4) are effective strategies for enhancing base pairing. These base substitutions enhance duplex stability and increase probe Tₘ by the following amounts: C-5 propynyl-C—2.8° C. per substitution; C-5 propynyl-U—1.7° C. per substitution. So-called BHQplus® provided by Biosearch technologies employ pdC and pdU substitutions in combination with BHQ dark quenchers. BHQplus and MGB probes can be used with oligonucleotides of shorter length and thus achieve an enhanced target specificity Another example of the probes used in real-time PCR assays are dual hybridization probes, which employ fluorescence resonance energy transfer (FRET) between the fluorophores on two different probes. Two fluorophore-labeled sequence-specific probes are designed to bind to the PCR product during the annealing phase of PCR, which results in an energy transfer from a donor fluorophore to an acceptor fluorophore. This results in an increase in fluorescence during the annealing phase. Some other examples of suitable probes are ZEN® Double-Quenched Probes (manufactured by Integrated DNA Technologies, Coraville, Iowa) (illustrated in FIG. 3) and QSY® probe from ThermoFisher Scientific, Waltham, Mass.

The terms “sample” or “samples,” as used interchangeably herein, include samples originating from human or animal subject (such as, but not limited to, samples of human or animal cells, tissues or bodily fluids and excretions) as well as samples prepared or generated by various laboratory and industrial processes, such as samples of virus isolates and vaccine samples. A sample can be directly obtained from a human or animal organism, obtained from the environment (such as food samples, water samples, surface swabs) propagated, cultured, synthesized or otherwise artificially produced. For example, a sample can be a virus isolate, including a primary isolate from a sample obtained from an infected individual, or an isolate propagated in the laboratory or industrially using various techniques, including recombinant techniques, tissue culture, propagation in eggs or nonhuman animals. Samples can be subject to various purification, storage or processing procedures before being analyzed according to the methods described herein. Samples can also be obtained at various steps of laboratory reactions and assays. For example, a sample may be produced by a PCR reaction. The methods described in this document may involve several different samples at different steps of the method. For example, a first sample may subjected to a PCR amplification step, and a second sample may be obtained after a PCR amplification step and subsequently processed or analyzed in a subsequent detection or analysis step or steps. Generally, the terms “sample” or “samples” are not intended to be limited by their source, origin, manner of procurement, treatment, processing, storage or analysis, or any modification.

The term “region” can be used in this document to refer to a part of a nucleic acid sequence, a part of a gene, or a part of a segment of influenza virus nucleic acid, and can be used interchangeably with the term “sequence.” For example, a region may be a part of a gene segment of influenza virus, such as a part of HA gene. For example, a region may be an HA segment of InfB virus corresponding to a region located approximately between nucleotides 229-295 in the sequence of HA InfB strain B/Nevada/03/2011 (National Center for Biotechnology Information (NCBI) access No. KC813804). A region may also be a region of an HA segment of InfB virus corresponding to a region located approximately between nucleotides 229-293 in the sequence of HA InfB strain B/Wisconsin/01/2010 (National Center for Biotechnology Information (NCBI) access No. JN993031).

The terms “sensitivity” and “specificity” can be used to refer to statistical measures of the performance of assays and methods described in this document. Sensitivity refers to a proportion of positive results which are correctly identified by a test. Specificity measures a proportion of the negative results that are correctly identified by a test. Examples of the calculations used to determine specificity and specificity are below.

sensitivity=(number of samples determined as positive by rRT-PCR assay)/(samples determined as positive by the standard test,such as sequence analysis)

specificity=(number of samples negative by rRT-PCR assay)/(samples determined as negative by the standard test,such as sequence analysis)

Limit of detection (LOD) refers to the ability of an assay to detect a target analyte (such as an influenza virus nucleic acid sequence), which is usually expressed as the minimum detectable concentration of the analyte. LOD can be expressed as a concentration of analyte, expressed in appropriate units describing a minimum concentration that can be detected by an assay or a detection method.

The term “sequence” can be used to refer to the order of nucleotides in a nucleic acid, which can also be described as “primary structure,” or to a nucleic acid molecule, such as an oligonucleotide, with a particular base order.

“Sequence identity” or “sequence similarity” in the context of two or more nucleic acids sequences, refer to two or more sequences or subsequences that are the same or have a specified percentage nucleotides that are the same (for example, 60%, 65%, 70%, 75%, 80%, 85%, 90%, 91%, 92%, 93%, 94%, 95%, 96%, 97%, 98%, 99%, or higher identity) over a specified region, when compared and aligned for maximum correspondence over a comparison window or designated region. Various tools for measuring sequence similarity are available, such as a BLAST or BLAST 2.0 sequence comparison algorithms with default parameters available from NCBI or other sources. See also Altschul et al., Nuc. Acids Res. 25:3389-3402 (1977) and Altschul et al., J. Mol. Biol. 215:403-410 (1990). For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

“Target nucleic acid” is a nucleic acid molecule or sequence intended for one or more of amplification, detection, quantitation, quantitative, semi-quantitative or qualitative detection. The nucleic acid molecule need not be in an isolated form purified state. Various other nucleic acid molecules can also be present with the target nucleic acid molecule. For example, the target nucleic acid molecule can be a specific nucleic acid sequence. It can include RNA (such as viral RNA) or DNA (such as DNA produced by reverse transcription of viral RNA). In the context of the embodiments of the present invention, a target nucleic molecule can be a nucleic acid sequence corresponding to a region of HA gene of InfB virus.

The embodiments of the present invention use PCR methods to detect target nucleic acids of influenza virus. “Quantitative PCR” is a method that allows for quantification of the amounts of the target nucleic acid sequence used at the start at the PCR reaction. “Real-time PCR” is a method for detecting and measuring products generated during each cycle of a PCR, which are proportionate to the amount of template nucleic acid prior to the start of PCR. The information obtained, such as an amplification curve, can be used to determine the presence of a target nucleic acid (such as an influenza virus nucleic acid) and/or quantitate the initial amounts of a target nucleic acid sequence. In some examples, real-time PCR is real time reverse transcriptase PCR (rRT-PCR). Although some sources use the terms “real-time PCR” and “quantitative PCR” synonymously, this is not the case for the present document. Here, the term “quantitative PCR” encompasses all PCR-based techniques that allow for quantification of the initially present target nucleic acid sequences. The term “real-time PCR” is used to denote a subset of quantitative PCR techniques that allow for detection of PCR product throughout the PCR reaction, or in real-time. The principles of real-time PCR are generally described, for example, in Held et al. “Real Time Quantitative PCR” Genome Research 6:986-994 (1996). Generally, real-time PCR measures a signal at each amplification cycle. Some real-time PCR techniques rely on fluorophores that emit a signal at the completion of every multiplication cycle. Examples of such fluorophores are fluorescence dyes that emit fluorescence at a defined wavelength upon binding to double-stranded DNA, such as SYBR green. An increase in double-stranded DNA during each amplification cycle thus leads to an increase in fluorescence intensity due to accumulation of PCR product. Another example of fluorophores used for detection in real-time PCR are sequence-specific fluorescent reporter probes, described elsewhere in this document. The examples of such probes are TaqMan® probes. The use of sequence-specific reporter probe provides for detection of a target sequence with high specificity, and enables quantification even in the presence of non-specific DNA amplification. Fluorescent probes can also be used in multiplex assays—for detection of several genes in the same reaction—based on specific probes with different-colored labels. For example, a multiplex assay can use several sequence-specific probes, labeled with a variety of fluorophores, including, but not limited to, FAM, JA270, CY5.5, and HEX, in the same PCR reaction mixture.

Real-time PCR relies on detection of a measurable parameter, such as fluorescence, during the course of the PCR reaction. The amount of the measurable parameter is proportional to the amount of the PCR product, which allows one to observe the increase of the PCR product “in real time.” Some real-time PCR methods allow for quantification of the input DNA template based on the observable progress of the PCR reaction. The analysis and processing of the data is discussed below. A “growth curve” or “amplification curve” in the context of a nucleic acid amplification assay is a graph of a function, where an independent variable is the number of amplification cycles and a dependent variable is an amplification-dependent measurable parameter measured at each cycle of amplification, such as fluorescence emitted by a fluorophore. As discussed above, the amount of amplified target nucleic acid (such as an influenza nucleic acid) can be detected using a fluorophore-labeled probe. Typically, the amplification-dependent measurable parameter is the amount of fluorescence emitted by the probe upon hybridization, or upon the hydrolysis of the probe by the nuclease activity of the nucleic acid polymerase. The increase in fluorescence emission is measured in real time and is directly related to the increase in target nucleic acid amplification (such as influenza nucleic acid amplification). In some examples, the change in fluorescence (dRₙ) is calculated using the equation dRₙ=Rₙ₊−Rₙ₋, with Rₙ₊ being the fluorescence emission of the product at each time point and Rₙ₋ being the fluorescence emission of the baseline. The dRₙ values are plotted against cycle number, resulting in amplification plots. In a typical polymerase chain reaction, a growth curve contains a segment of exponential growth followed by a plateau, resulting in a sigmoidal-shaped amplification plot when using a linear scale. A growth curve is characterized by a “cross point” value or “Cₚ” value, which can be also termed “threshold value” or “cycle threshold” (Cₜ), which is a number of cycles where a predetermined magnitude of the measurable parameter is achieved. For example, when a fluorophore-labeled probe is employed, the threshold value (Cₜ) is the PCR cycle number at which the fluorescence emission (dRₙ) exceeds a chosen threshold, which is typically 10 times the standard deviation of the baseline (this threshold level can, however, be changed if desired). A lower Cₜ value represents more rapid completion of amplification, while the higher Cₜ value represents slower completion of amplification. Where efficiency of amplification is similar, the lower Cₜ value is reflective of a higher starting amount of the target nucleic acid, while the higher Cₜ value is reflective of a lower starting amount of the target nucleic acid. Where a control nucleic acid of known concentration is used to generate a “standard curve,” or a set of “control” Cₜ values at various known concentrations of a control nucleic acid, it becomes possible to determine the absolute amount of the target nucleic acid in the sample by comparing Cₜ values of the target and control nucleic acids.

Assays

Embodiments of the present invention include real-time RT-PCR (rRT-PCR) assays useful for lineage-specific detection InfB viruses, meaning that the assays specifically detect InfB viruses of Victoria or Yamagata lineages. Thus, the assays according to the embodiments of the present invention can be referred to as “lineage” assays. The primers of the InfB lineage assay are useful for amplification of a region of InfB HA gene specific for Yamagata or Victoria lineages. The probes are used for specific detection of the amplification products. When contacted in the sample in the context of the rRT-PCR assay, the primers amplify a section of the HA1 domain of HA gene of type B influenza virus, and the probes target Yamagata- or Victoria-specific sequences within the amplified section, generating a signal that can be interpreted to determine the presence or absence of Yamagata or Victoria-lineage InfB HA sequences in the sample. An example of an InfB lineage assay employs a forward primer, a reverse primer and a probe labeled with a fluorescent label and a dark quencher for specific detection of InfB viruses belonging to Victoria lineage (“VIC assay”), and a different forward primer, reverse primer and probe for specific detection of Yamagata lineage InfB viruses (“YAM assay”).

Specificity of the InfB lineage assays according to the embodiments of the present invention allows them to discriminate between Yamagata and Victoria InfB virus strains. When the assays of the present invention are used to detect InfB virus of the specified lineage, the specificity of the assays according to the embodiments of the present minimizes or avoids false positive assay results generated by other types lineage assays. Sensitivity of the InfB lineage assays according to the embodiments of the present invention allows them to avoid false negative assay results. LOD of the InfB lineage assays according to the embodiments of the present invention allows them to detect low amounts of InfB nucleic acids. Specificity of the assays according to the embodiments of the present invention can be at least about 95%, 96%, 97%, 98%, or about 95%, 96%, 97%, 98%, or 100%. Sensitivity of the assays according to the embodiments of the present invention can be at least about 95%, 96%, 97%, 98%, or about 95%, 96%, 97%, 98%, or 100%. LOD of the assays according to the embodiments of the present invention can be about 10^{4.0}, 10^{3.5}, 10^{3.0}, 10^{2.5}, 10^{2.4}, 10^{2.3}, 10^{2.2}, 10^{2.1}, 10^{2.0} or less than any of the above values denoting Egg Infectious Doses per milliliter (EID₅₀/ml).

InfB lineage assays of the present invention are designed to detect a region of HA gene of InfB virus strain using one or more DNA primers and probes based on the sequences listed in Table 1. It is to be understood, of course, that the uses of assays of the present invention, as well as of the primers and the probes described in this documents, are not limited to the detection of InfB virus strains. The assays, the primers and the probes can be used to detect any influenza virus or, more generally, the nucleic acids containing one or more the relevant sequences, or their variants, which were used in the primer and/or probe design (shown in Table 1).

The assays according to the embodiments of the present invention can serve as effective tools for rapid and specific identification InfB of viruses of Yamagata and Victoria lineages in clinical and laboratory samples with high sensitivity, specificity and superior LOD. The assays of the present invention can have various application and uses. For example, current trivalent human influenza vaccines contain a single InfB component representing the predominant circulating lineage. In order to make appropriate vaccine recommendations, influenza surveillance information needs to be gathered accurately and timely to determine what the predominant circulating InfB lineage is. The assays based on virus culture hemagglutination inhibition (HI), microneutralization, conventional RT-PCR, restriction fragment length polymorphism (RFLP), and nucleotide sequencing assays are cumbersome and time-consuming. The assays based on sequencing and microarrays are too technologically advanced and costly for worldwide use. InfB lineage assays according to the embodiments of the present invention are accurate, quick and technologically accessible. They can be used to test samples collected from individuals with respiratory symptoms to determine which InfB lineage has been a causative agent. InfB lineage assays can also be employed to test InfB vaccine samples to verify their identity and the titer of InfB virus.

TABLE 1
Sequences used in the primer and probe design

| | Sequence | Location in |
|---|---|---|
| Sequence name | (5′ > 3′) | HA sequence |
| InfB Victoria Lineage | GAT CTG GAC GTA | 229-248 |
| Forward Primer 1 | GCC TTG GG | |
| (VIC forward primer 1) | SEQ ID NO: 1 | |
| InfB Victoria Lineage | GAT CTG GAT GTA | 229-248 |
| Forward Primer 2 | GCC TTG GG | |
| (VIC forward primer 2) | SEQ ID NO: 7 | |
| InfB Victoria Lineage | TAA CAG GTC TGA | 316-295 |
| Reverse Primer | CTT CAT GGA G | |
| (VIC reverse primer) | SEQ ID NO: 2 | |
| InfB Victoria lineage | TTC CCC GTG CAT | 269-254 |
| Probe 1 (VIC probe 1) | TTT G | |
| | SEQ ID NO: 3 | |
| InfB Victoria lineage | TTC CCC GTG CAT | 269-254 |
| Probe 2 (VIC probe 2) | TTT G | |
| | SEQ ID NO: 8 | |
| InfB Yamagata Lineage | GAT CTG GAT GTG | 229-248 |
| Forward Primer | GCC TTG GG | |
| (YAM forward primer) | SEQ ID NO: 4 | |
| InfB Yamagata Lineage | AGG TCT GAC YTC | 311-293 |
| Reverse Primer 1 | GTG RAG TA | |
| (YAM reverse primer 1) | SEQ ID NO: 5 | |
| InfB Yamagata Lineage | AC AGG TCT GAC | 309-292 |
| Reverse Primer 2 | YTC ATG GAG TAT | |
| (YAM reverse primer 2) | SEQ ID NO: 9 | |
| Inf B Yamagata Probe | CAC ACA CAT TGG | 264-250 |
| (YAM probe) | CCT | |
| | SEQ ID NO: 6 | |

^{a}Location numbering based on first base of the coding region for the Influenza B Hemagglutinin 1 protein of InfB-Victoria-Like strain B/Nevada/03/2011 (NCBI Accession: KC813804) and InfB-Yamagata-like strain B/Wisconsin/01/2010 (NCBI Accession: JN993031)

The assays according to the embodiments of the present invention can be singleplexassay or multiplex assays. For example in singleplex rRT-PCR assay, only InfB Yamagata primers and probe or only InfB Victoria lineage primers and probe are used to amplify and detect InfB HA gene sequence sequences, respectively. In other words, a YAM assay or a VIC assay can be performed as a singleplex assay. The data obtained from a set of singleplex assays can be analyzed to make various diagnostic determination. For example, a YAM assay, a VIC assay and universal InfB assay (used to detect InfB virus in a sample, regardless of its lineage) can be performed as singleplex assays on aliquots of the same sample to detect InfB and to determine InfB lineage. InfB universal assay is used in this situation to provide additional diagnostic assurance. In contrast, in a multiplex rRT-PCR assay, both InfB Yamagata primers and probe and InfB Victoria lineage primers and probe can be used in a single reaction to amplify and detect Yamagata and Victoria InfB HA gene sequence sequences potentially present in the sample. In other words, a YAM assay and a VIC assay can be performed together as a multiplex assay. The assays according to the embodiments of the present invention can also be multiplexed with other assays. For example, a YAM assay, a VIC assay or both YAM and VIC assay can be multiplexed with other influenza virus assays. In one embodiment, a YAM assay, a VIC assay or both YAM and VIC assay can be multiplexed with an InfB universal assay and performed on the same sample. A YAM assay, a VIC assay or both YAM and VIC assays can also be multiplexed with other influenza assays or assays for detecting non-influenza pathogens. In a multiplex assay, the probes and/or the primers are uniquely labelled to distinguish their signals. Multiplex assays allow one to measure the expression levels of several targets or genes of interest quickly. Multiplex assays also minimize the amount of starting material required, which can be of critical value when samples are limited. Multiplexing can also save time by increasing throughput and decreasing sample handling. It can also save on the cost of reagents and other consumables.

Probes

Some embodiments of the present invention are oligonucleotide probes that can be employed to detect InfB virus in rRT-PCR assays. The probes of the present invention are designed to detect HA1 domain region of HA gene of InfB influenza virus and are based on the sequences listed in Table 1. It is understood that the probe sequences are not limited to SEQ ID NOs 3 and 6, but can include their variations, which can be defined based on sequence similarity. It is also understood that the probes are not limited to detection of InfB virus and can be used to detect any influenza virus nucleic acids or other nucleic acids containing the sequences used in the probe design (shown in Table 1) or their variants. The use of the probes is not limited to rRT-PCR assays, they can be used in other assays and methods, such as array detection.

The embodiments of the present invention include DNA probes suitable for detection of a region of HA gene of InfB virus of Victoria lineage, which contain SEQ ID NO:3 or its variant, such as SEQ ID NO:8. Embodiment of such probes can be referred to as VIC probes. Some embodiments of the probe contain (comprise) an oligonucleotide at least 85% identical to SEQ ID NO:3 (for example, an oligonucleotide at least 90% identical to SEQ ID NO:3, an oligonucleotide at least 95% identical to SEQ ID NO:3, or an oligonucleotide of SEQ ID NO:3). Some other embodiments consist of an oligonucleotide at least 85% identical to SEQ ID NO:3 (for example, an oligonucleotide at least 90% identical to SEQ ID NO:3, an oligonucleotide at least 95% identical to SEQ ID NO:3 or an oligonucleotide of SEQ ID NO:3) and reporting moieties discussed elsewhere in this document. Some example of VIC probes are SEQ ID NOs 3 or 8 with reporting moieties, such as FAM at 5′ end and BHQ1 at 3′ end.

The embodiments of the present invention include DNA probes suitable for detection of a region of HA gene of InfB virus of Yamagata lineage, which contain SEQ ID NO:6 or its variants. Embodiments of such probes can be referred to as YAM probes. Some embodiments of the probe contain (comprise) an oligonucleotide at least 85% identical to SEQ ID NO:6 (for example, an oligonucleotide at least 90% identical to SEQ ID NO:6, an oligonucleotide at least 95% identical to SEQ ID NO:6 or an oligonucleotide of SEQ ID NO:6). Some other embodiments consist of an oligonucleotide at least 85% identical to SEQ ID NO:6 (for example, an oligonucleotide at least 90% identical to SEQ ID NO:6, an oligonucleotide at least 95% identical to SEQ ID NO:6 or an oligonucleotide of SEQ ID NO:6) and reporting moieties discussed elsewhere in this document. An example of a YAM probe is SEQ ID NO:6 with reporting moieties, such as FAM at 5′ end and BHQ1 at 3′ end.

The length of a probe depends on the primers selected for a particular rRT-PCR assay and other factors, such as probe chemistry. An exemplary probe can be 12-30 bp long. For example a probe can be 12-18 bp long, about 15 bp long (meaning 15±3, 15±2, 1±1 bp long) 12, 13, 14, 15, 16, 17 or 18 bp long). A probe is designed with about 8-10° C. higher Tₘ than T_{mS} of the primers. A probe typically contains reporting moieties and may contain other moieties, such as linkers, stabilizers, modified bases, etc., the selection of which depends on a probe chemistry. In one example, the probe can be a TaqMan® problem labeled with a fluorophore moiety, such as FAM, and a quencher moiety, but other types of probe chemistries can be employed. In an exemplary TaqMan® probe, the fluorophore moiety is coupled to 5′ terminus of the probe. One example of a suitable fluorophore is a fluorescein moiety. One example of a suitable quencher is a dark quencher, for example BHQ quencher, such as BHQ1. The quencher can be coupled to 3′ terminus of the probe or to an internal base. The probe can also contain a duplex stabilizer.

One exemplary embodiment of a VIC probe is a probe consisting of SEQ ID NO:3 oligonucleotide, FAM fluorophore coupled to 5′ terminus and BHQ1 quencher coupled to 5′ end of the oligonucleotide, as shown in Table 2. Another exemplary embodiment of a VIC probe is a probe consisting of SEQ ID NO:8 oligonucleotide, FAM fluorophore coupled to 5′ terminus and BHQ1 quencher coupled to 5′ end of the oligonucleotide, as shown in Table 2. One exemplary embodiment of a YAM probe is a probe consisting of SEQ ID NO:6 oligonucleotide, FAM fluorophore coupled to 5′ terminus and BHQ1 quencher coupled to 5′ end of the oligonucleotide, as shown in Table 2. Some embodiments of the probes incorporate MGB moieties and/or modified bases. The ZEN® Double-Quenched Probes (manufactured by Integrated DNA Technologies, Coraville, Iowa) and QSY® probe from ThermoFisher Scientific, Waltham, Mass. comprising SEQ ID NO:3 oligonucleotide for VIC probes and SEQ ID NO:6 oligonucleotide for VIC probes It is to be understood that the choice of a fluorophore and quencher for a particular probe depends on the type of a probe and probe design.

Primers

Embodiments of the present invention include DNA oligonucleotides that can be employed for amplification of InfB virus HA gene segment sequences. The uses of the primers described in this document are not limited to InfB sequence amplification; the primers can be used to amply any nucleic acids containing regions having sequence similarity to the primer sequences shown in Table 1. The uses of the primers according to the embodiments of the present invention are also not limited to PCR amplification, such as rRT-PCR assays; the primers can be used in various other assays and methods, for example, sequencing or array-based detection.

The primes according to the embodiments of the present invention are based on SEQ ID NOs 1, 2, 4, 5, 7 or 9, which are shown in Table 1. Some embodiments of the primers contain (comprise) an oligonucleotide at least 85% identical to SEQ ID NOs 1, 2, 4 or 5 (for example, an oligonucleotide at least 90% identical to SEQ ID NOs 1, 2, 4 or 5, an oligonucleotide at least 95% identical to SEQ ID NOs 1, 2, 4 or 5, an oligonucleotide 99% identical to SEQ ID NOs 1, 2, 4 or 5, or an oligonucleotide of SEQ ID NOs 1, 2, 4, 5, 7 or 9. Some other embodiments consist of an oligonucleotide at least 85% identical to SEQ ID NOs 1, 2, 4 or 5 (for example, an oligonucleotide at least 90% identical to SEQ ID NOs 1, 2, 4 or 5, an oligonucleotide at least 95% identical to SEQ ID NOs 1, 2, 4 or 5, an oligonucleotide at least 99% identical to SEQ ID NOs 1, 2, 4 or 5, or an oligonucleotide of SEQ ID NOs 1, 2, 4, 5, 7 or 9). Some examples of such primers are SEQ ID NOs 1, 2, 4, 5, 7 or 9, or oligonucleotides comprising SEQ ID NOs 1, 2, 4, 5, 7 or 9.

Among the embodiments of the present invention are primers suitable for amplification of a region of a region of HA gene of InfB virus of Victoria lineage (“Victoria primers”). Victoria primers include a “forward” primer comprising an oligonucleotide at least 90% identical SEQ ID NO:1 (“VIC forward primer”), for example, a primer of SEQ ID NOs 1 or 7, and a “reverse” primer comprising an oligonucleotide at least 90% identical SEQ ID NO:2 (“VIC reverse primer”). For example, a VIC forward primer can be an oligonucleotide comprising SEQ ID NO:1 or its variant, such as SEQ ID NO:7, an oligonucleotide consisting of SEQ ID NO:1 or its variant, such as SEQ ID NO:7, or oligonucleotide consisting of SEQ ID NO:1 or its variant, such as SEQ ID NO:7, and optional reporting moieties or labels. A VIC reverse primer can be an oligonucleotide comprising SEQ ID NO:2 or its variant, an oligonucleotide consisting of SEQ ID NO:2 or its variant, or oligonucleotide consisting of SEQ ID NO:2 or its variant and optional reporting moieties or labels. It is understood that, for amplification of a region of HA1 gene of InfB virus or other uses, Victoria primers can be used together as a primer pair, but can also be used separately in combination with the other primers. For example, VIC forward primer can be combined with VIC reverse primer for amplification of InfB HA gene region, but can also be combined with a suitable primer other than VIC reverse primer. Likewise, VIC reverse primer can be combined with VIC forward primer for amplification of InfB HA gene region of InfB, but can also be combined with a suitable primer other than VIC forward primer.

Also among the embodiments of the present invention are primers suitable for amplification of a region of a region of HA gene of InfB virus of Yamagata lineage (“Yamagata primers”). Yamagata primers include a “forward” primer comprising an oligonucleotide at least 85% (for example, 90% or 95%) identical to SEQ ID NO:4 (“YAM forward primer”) and a “reverse” primer comprising an oligonucleotide at least 85% (for example, 90% or 95%) identical to SEQ ID NO:5, such as SEQ ID NO:9 (“YAM reverse primer”). For example, a VIC forward primer can be an oligonucleotide comprising SEQ ID NO:4 or its variant, an oligonucleotide consisting of SEQ ID NO:4 or its variant, or oligonucleotide consisting of SEQ ID NO:4 or its variant and optional reporting moieties or labels. A YAM reverse primer can be an oligonucleotide comprising SEQ ID NO:5 or its variant, such as SEQ ID NO:9, an oligonucleotide consisting of SEQ ID NO:5 or its variant, such as SEQ ID NO:9, or oligonucleotide consisting of SEQ ID NO:5 or its variant, such as SEQ ID NO:9, and optional reporting moieties or labels. It is understood that, for amplification of region of a region of HA1 gene of InfB virus or other uses, Yamagata primers can be used together as a primer pair, but can also be used separately in combination with the other primers. For example, YAM forward primer can be combined with YAM reverse primer for amplification of HA gene region, but can also be combined with a suitable primer other than YAM reverse primer. Likewise, YAM reverse primer can be combined with VIC forward primer for amplification of HA gene region of InfB, but can also be combined with a suitable primer other than YAM forward primer.

It is to be understood that the primers according to the embodiments of the present invention can be unmodified and unlabeled DNA oligonucleotides. The primers according to the embodiments of the present invention can also contain reporting or labelling moieties, such as fluorescent moieties, quencher moieties or their combinations. The primers according to the embodiments of the present invention can also contain unnatural and modified nucleotides, linkers and other moieties. The length of the primers can vary. For example, the primers can be 15-30 bp long. A primer length is selected to be long enough for adequate specificity and short enough for primers to bind easily to the target nucleic acid at the annealing temperature. For example, the primers can be 20-30 bp long, for example, 20, 21, 22, 23, 24, 35, 26, 27, 28, 29, 30, 31 and 32 bp long. A primer is designed to have a Tₘ that is 8-10° C. lower than Tₘ of the probe, yet sufficiently high to ensure specific binding. An exemplary primer can have a Tₘ of about 55-60° C., for example, about 58, 59 or 60° C., but T_{mS} outside of this range are also possible, depending on the specific primer.

### Kits

The embodiments of the present invention also include kits comprising one or more of the primers and the probes described above. In other words, the primers according to the embodiments of the present invention can be included or combined, in various ways, in kits. Such kits can be used for detection, including semi-quantitative and quantitative detection, of Yamagata lineage InfB viruses, Victoria lineage InfB viruses, or both Yamagata and Victoria InfB virus strains in samples, such as the samples derived from human or animal subjects, laboratory samples, virus isolate samples or vaccine samples. It is to be understood that at least some of the kits described in this document are not limited to InfB amplification or detection and can be used to detect and/or amplify any influenza virus nucleic acids or other nucleic acids containing the sequences used in the design or the probes included in the kits. These sequences are shown in Table 1.

Some examples of the kit embodiments are described below. YAM or VIC probes or both YAM and VIC probes can be included in the kits useful for detecting or differentiating InfB virus strains by rRT-PCR assays. For example, a YAM probe can be included in a kit along with other reagents for performing an rRT-PCR assay. Such a kit can be used for detecting Yamagata InfB virus strain in the sample. In another example, a VIC probe can be included in a kit along with other reagents for performing an rRT-PCR assay. Such a kit can be used for detecting Victoria InfB virus strain in the sample. In one more example, a YAM probe and a VIC probe can be included in a kit along with other reagents for performing an rRT-PCR assay. Such a kit can be used for detecting Yamagata InfB virus, Victoria InfB virus or both in the sample.

The other reagents included in the kits can include one or more Victoria and Yamagata primers. For example, a kit can include a YAM probe and one or both of YAM forward primer and YAM reverse primer. In another example, a kit can include a VIC probe and one or both of VIC forward primer and VIC reverse primer. In one more example, a kit can include a VIC probe, a YAM probe, one or both of VIC forward primer and VIC reverse primer, and one or both of YAM forward primer and YAM reverse primer.

The kits can include additional reagents for performing an rRT-PCR assay. The examples of additional reagents are enzymes for performing rRT-PCR assays are reverse transcriptase, DNA polymerase, such as Taq polymerase, PCR buffers, dNTPs and various additives, such as the additives that allow for efficient amplification of GC-rich templates. Some other examples of possible additional reagents are DNA-binding dyes, such as SYBR Green, which can be employed in rRT-PCR assays that employ unlabeled primers and no probes.

Methods

Embodiments of the present invention also include methods of using the primers, probes and kits described above (“method embodiments”). Some of the method embodiments are methods of amplifying a region of a HA gene of InfB virus by a PCR using one or more of the primers described in this document. Such methods can be referred to as “methods of amplifying an InfB virus strain,” “methods of amplifying an InfB virus sequence,” “methods of amplifying a region of HA sequence of InfB virus” “amplification methods,” and by other related expressions and include a step of contacting a sample, which may contain an InfB HA nucleic acid sequences, with one or more primers described in this document. When the goal of the method is amplifying a region of a HA gene of Yamagata lineage InfB virus, a forward YAM primer, a reverse YAM primer, or a combination of forward and reverse YAM primers is employed. When the goal of the method of the method is amplifying a region of a HA gene of Victoria lineage InfB virus, a forward VIC primer, a reverse VIC primer, or a combination of forward and reverse VIC primers is employed. It is to be understood that both YAM and VIC primers in various combinations can be employed in some embodiments of the amplification methods. After the contacting step, a PCR (such as rRT-PCR, discussed in more detail elsewhere in this document) is performed under suitable conditions and using suitable reagents, and the amplification products can be detected by various detection procedures. The amplification methods can be used to determine if a nucleic acid sequence corresponding to Yamagata and/or Victoria InfB virus strain is present in the sample, based on the detection of one or more products of the amplification.

Some of the method embodiments rely on detection of a gene region of HA gene of Yamagata InfB virus strain and/or detection of a gene region of HA gene of Victoria InfB virus strains using the probes according to the embodiment of the present invention in a rRT-PCR assay. One example of a method embodiment, which can be referred to as “detection method” or “method of detecting” is a method of detecting a presence or absence of a Yamagata InfB virus strain in a sample. The detection method embodiment includes a step of contacting a sample with a YAM InfB probe described in this document. The method embodiment can also include a step of contacting a sample and forward and reverse primers specific for at least one nucleic acid sequence of the HA gene region of InfB for which the probe is specific. A forward primer may be one of the YAM primers described in this document. A reverse primer may be one of the YAM primers described in this document. Another example of a method embodiment, which can be referred to as “detection method” or “method of detecting” is a method of detecting a presence or absence of a Victoria InfB virus strain in a sample. The detection method embodiment includes a step of contacting a sample with a VIC InfB probe described in this document. The method embodiment can also include a step of contacting a sample and forward and reverse primers specific for at least one nucleic acid sequence of the HA gene region of InfB for which the probe is specific. A forward primer may be one of the VIC primers described in this document. A reverse primer may be one of the VIC primers described in this document.

In the detection methods that employ rRT-PCR (rRT-PCR methods or assays), rRT-PCR is performed under suitable conditions and using suitable reagents following the contacting step in order to generate a PCR cycle threshold, and this cycle threshold is compared to a control value. In a semi-quantitative variation of rRT-PCR methods, if the cycle threshold is below the control value, the InfB virus strain sequence is absent from the sample, and if the cycle threshold is above the control value, the InfB virus strain sequence is present in the sample. An example of a cycle threshold control (cutoff) value is Cₜ value of CDC Human Influenza Real-Time RT-PCR Diagnostic Panel, Cₜ=38. In a quantitative variation of rRT-PCR methods, the method can include a step of determining a quantity of the InfB virus strain being detected (for example, a Victoria or Yamagata lineage strain) when the InfB virus strain is present in the sample.

The calculations and comparisons (for example, of a sample signal to a control value or range) for the methods described in this document can involve computer-based calculations and tools. Tools can be advantageously provided in the form of computer programs that are executable by a general purpose computer system (which can be called “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (for example, using PERL), execution of which can be initiated by a user from a standard command line interface of an operating system such as Linux or UNIX. Commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

The amplification and the detection methods of the present invention can have various applications. For example, they can be used in a method of determining if a human or animal subject is infected with a particular lineage of InfB virus strain, meaning InfB having a gene region from HA gene of Yamagata or Victoria InfB virus strain. Such a method can be employed as a surveillance method to determine, for example, which lineage of InfB strain circulates in the community and to make the decisions about the type of InfB strain to be included in the influenza vaccine for community distribution. In another example, testing of a collection of the samples obtained from a population using the methods of the present invention can generate more accurate epidemiological data on circulation of InfB virus strains. The present invention, thus can provide an important contribution to public health surveillance, clinical diagnosis and scientific investigations to differentiate influenza B positive specimens as Yamagata or Victoria lineage.

The amplification and the detection methods of the present invention can also be used for quality control of influenza vaccines. For example, influenza vaccine samples, particularly, but not limited, those originating from suspect sources or suspected of being exposed suboptimal storage or production conditions, can be tested to verify the identify the presence and the amounts of InfB virus strains found in the vaccines.

### EXAMPLES

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### Propagation of Influenza Virus Isolates and Nucleic Acid Extraction

Influenza viruses were propagated in either Madin-Darby Canine Kidney (MDCK) cells or 10-11 day old embryonated chicken eggs (ECE), using the methods described in Szretter, K. J et al., “Influenza: propagation, quantification, and storage.” Curr Protoc Microbiol, 2006. Chapter 15: p. Unit 15G 1. Virus concentrations were estimated by determining a 50% infectious dose (ID₅₀/mL) in either tissue culture supernatant (TCID₅₀/ml) or ECE allantoic fluid (EID₅₀/mL), respectively, using the method described in Reed, L. J., “A simple method of estimating fifty percent endpoints.” American Journal of Epidemiology, 1938. 27(3): p. 493. VIC lineage InfB viruses used for analytical performance evaluation were represented by B/Brisbane/60/2008 and B/Nevada/03/2011, and YAM lineage viruses were represented by B/Wisconsin/01/2010 and B/Texas/06/2011. InfB viral isolates used for analytical inclusivity testing are shown in Table 3. InfB virus lineages were confirmed using antigenic characterization by hemagglutination inhibition assay (HI), described in Lindstrom, S. E., et al., “Comparative analysis of evolutionary mechanisms of the hemagglutinin and three internal protein genes of influenza B virus: multiple cocirculating lineages and frequent reassortment of the NP, M, and NS genes.” J. Virol., 1999. 73(5): p. 4413-26, and genetic sequence analysis. Influenza A viral isolates used for specificity testing are listed in Table 5. The results of the testing with non-influenza human respiratory viruses and bacteria are shown in Tables 6 and 7.

RNA was isolated from 100 μL of influenza viral isolates and non-influenza respiratory RNA viruses using the MagNA Pure Compact instrument with the RNA Isolation Kit (Roche Diagnostics, Mannheim, Germany) using the manufacturer's RNA_Tissue-V3.2 protocol. The final elution volume was 100 μL.

Total nucleic acid was extracted in the Roche MagNA Pure Compact instrument from non-influenza DNA respiratory viruses and bacteria using 100 μL of sample and the Total Nucleic Acid Kit (Roche Diagnostics, Mannheim, Germany) following the manufacturer's Total_NA_Plasma_100_400 V3.2 protocol. The final elution volume was 100 μL.

### Example 2

### Primers and Probes

The probes and the primers used in the tested InfB lineage assays are shown in Table 2. The assays were designed to be used with universal influenza B assay (InfB) (universal detection of the NS gene of influenza B viruses) from the CDC Flu rRT-PCR Dx Panel assay (“InfB Universal Assay”). A sample was considered positive for either VIC or YAM lineage if both InfB universal assay and InfB lineage assays generated positive result for either Yamagata or Victoria InfB virus.

The primers and the probes shown in Table 2 were designed using nucleotide sequences of hemagglutinin (HA) gene of historical and contemporary influenza B viruses available from NCBI and the GISAID EpiFlu™ databases using BioEdit biological sequence alignment editor (Ibis Biosciences Carlsbad, Calif.) and the Beacon Designer™ v6 software package (Premier Biosoft, Palo Alto, Calif.). BHQplus™ dual-labeled hydrolysis probes were designed using the Biosearch Technologies Real Time Design™ software package. The primers and the probes showed no potential cross-reactivity with other respiratory pathogens or human genome by NCBI BLAST analysis. The probes were labeled at the 5′-end with the reporter molecule 6-carboxyfluorescein (FAM) and incorporated BHQ1™ quencher supplied by Biosearch Technologies, Inc. (Novato, Calif.). The probes incorporating MGB moieties and modified bases were also produced and tested, producing the results comparable to those discussed further.

TABLE 2
Primers and probes

| | Sequence | | |
|---|---|---|---|
| Name | (5′ > 3′) | SEQ ID NO | Label |
| VIC forward | GAT CTG GAC | SEQ ID NO: 1 | None |
| primer 1 | GTA GCC TTG GG | | |
| VIC forward | GAT CTG GAT | SEQ ID NO: 7 | None |
| primer 2 | GTA GCC TTG GG | | |
| VIC reverse | TAA CAG GTC TGA | SEQ ID NO: 2 | None |
| primer | CTT CAT GGA G | | |
| VIC probe 1 | TTC CCC GTG CAT | SEQ ID NO: 3 | FAM at |
| | TTT G | | 5′ end; |
| | | | BHQ1 at |
| | | | 3′ end. |
| VIC probe 2 | TTC CCC GTG CAT | SEQ ID NO: 8 | FAM at |
| | TTT G | | 5′ end; |
| | | | BHQ1 at |
| | | | 3′ end. |
| YAM forward | GAT CTG GAT GTG | SEQ ID NO: 4 | None |
| primer | GCC TTG GG | | |
| YAM reverse | AGG TCT GAC YTC | SEQ ID NO: 5 | None |
| primer 1 | GTG RAG TA* | | |
| YAM reverse | AC AGG TCT GAC | SEQ ID NO: 9 | None |
| primer 2 | YTC ATG GAG TAT* | | |
| YAM probe | CAC ACA CAT TGG | SEQ ID NO: 6 | FAM at |
| | CCT | | 5′ end; |
| | | | BHQ1 at |
| | | | 3′ end. |

*R stands for purine, A or G; Y stands for pyrimidine, T or C; primer sequences were supplied with approximately 1:1 ratio of the ambiguous bases

### Example 3

### InfB Assay rRT-PCR Conditions

Optimal rRT-PCR thermocycling parameters were determined using Invitrogen SuperScript™ III Platinum One-Step QRT-PCR System (Invitrogen by Life Technologies, Carlsbad, Calif.) and Bio-Rad CFX96™ Real-time PCR System (Bio-Rad Laboratories, Hercules, Calif.). Thermal gradient analysis was performed under the following conditions: reverse transcription step of 50° C. for 30 min, Taq activation step of 95° C. for 2 min, 45 cycles of denaturation at 95° C. for 15 sec and an annealing/extension range of 50° C.-63° C. for 30 sec. The following thermal cycling parameters were used in the validation assays: reverse transcription step of 50° C. for 30 min, Taq activation step of 95° C. for 2 min, 45 cycles of denaturation at 95° C. for 15 sec and an annealing/extension range of 55° C. for 30 sec. The rRT-PCR reactions were performed using primer and probe reaction concentrations of 0.8 μM and 0.2 respectively, with a final volume of each reaction being 25 μL. All analytical performance data described below was collected using the Invitrogen SuperScript™ III Platinum One-Step QRT-PCR System on the Applied Biosystems 7500 Fast Dx Real-Time PCR Instrument (Applied Biosystems, Foster City, Calif.). The assays were also performed (and similar results achieved) using additional enzyme systems: qScript (Quantabio, Bevery, Mass.) and Agpath-ID™ (Thermo Fischer Scientific, Walham, Mass.). The assay thermocylcing parameters were similar to those described above, with the exception that Taq activation step was performed 95° C. for 5 min.

### Example 4

### Primer and Probe Performance

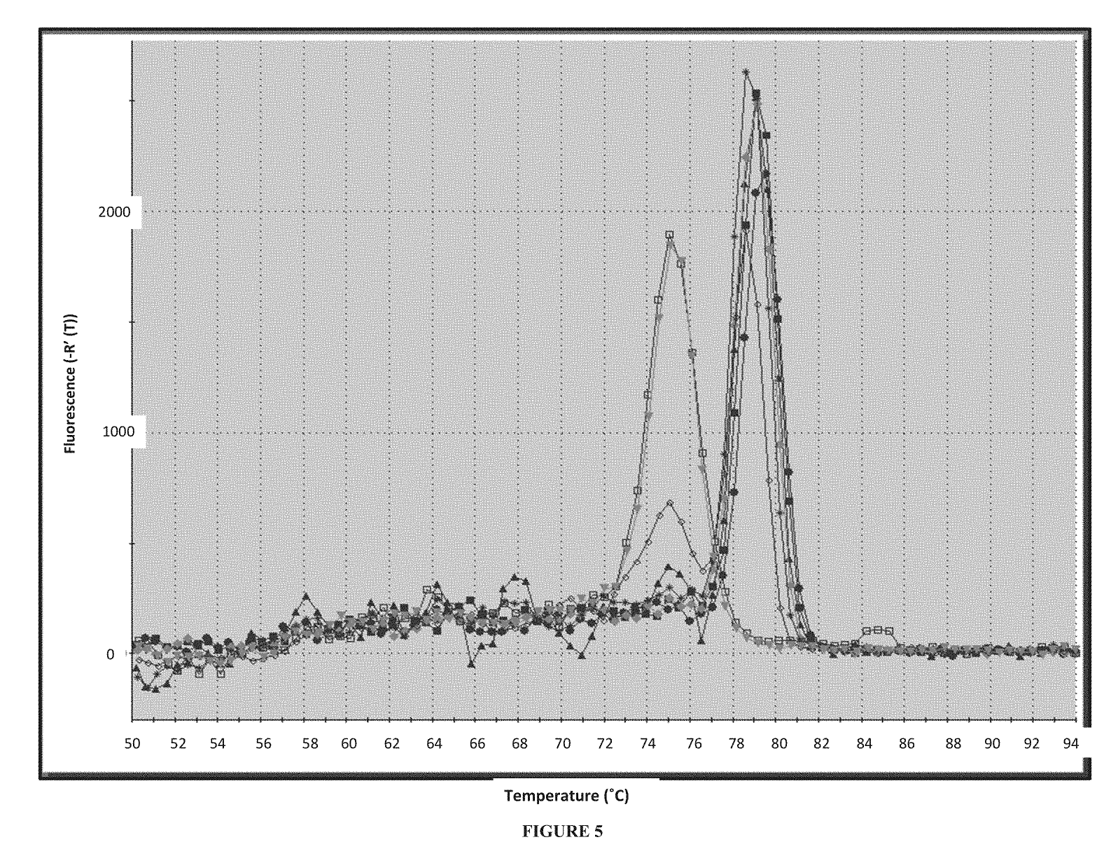

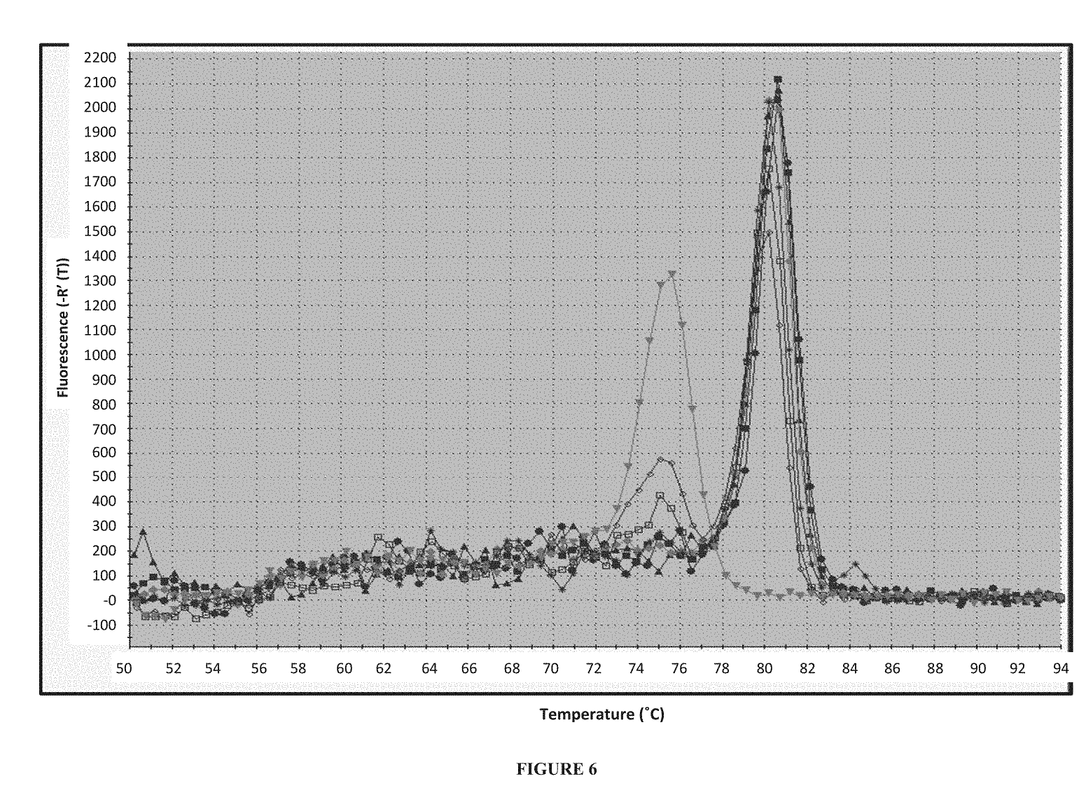

Primer performance was evaluated using melt-curve analysis. The melt-curve analysis was performed using QuantiTect™ SYBR® Green RT-PCR Kit (Qiagen, Inc., Valencia, Calif.) and Agilent Technologies Stratagene Mx3005P qPCR System (Agilent Technologies, Inc. Santa Clara, Calif.) with (1) 10-10 fold serial dilutions of B/Nevada/03/2011 B-Victoria-like viral RNA as a template and VIC forward primer 1 and VIC reverse primer (shown in Table 2), and (2) 10-fold serial dilutions of B/Wisconsin/01/2010 B-Yamagata-like RNA and YAM forward primer and YAM reverse primer 1 (shown in Table 2). The results of melt curve analysis are illustrated, respectively, in FIGS. 5 (VIC primers) and 6 (YAM primers). Melt-curve analysis revealed amplification of a single product of predicted size for both VIC and YAM primer pairs tested. Primer-dimer cross reaction was seen in small amounts in the no-template control reactions at the lowest concentration of viral RNA.

TABLE 3
Determination of optimal annealing temperature
A. VIC probe 1

| | Cₜ value | | | |
|---|---|---|---|---|
| | Inf B Universal Assay | | VIC probe 1 | |
| | High | Low | High | Low |
| Annealing | concentra- | concentra- | concentra- | concentra- |
| temperature | tion | tion | tion | tion |
| 50.0° C. | 20.26 | 29.76 | 19.11 | 28.76 |
| 50.8° C. | 20.10 | 29.74 | 19.17 | 29.11 |
| 52.6° C. | 20.23 | 29.50 | 20.24 | 29.89 |
| 55.1° C. | 19.93 | 30.17 | 19.80 | 30.49 |
| 58.2° C. | 19.96 | 29.33 | 20.75 | 31.24 |
| 60.8° C. | 20.21 | 29.75 | 21.50 | 32.25 |
| 62.3° C. | 20.06 | 29.66 | 22.01 | 33.18 |
| 63.0° C. | 19.91 | 29.66 | 22.03 | 33.38 |

B. YAM probe

| | Cₜ value | | | |
|---|---|---|---|---|
| | Inf B Universal Assay | | YAM probe | |
| | High | Low | High | Low |
| Annealing | concentra- | concentra- | concentra- | concentra- |
| temperature | tion | tion | tion | tion |
| 50.0° C. | 20.28 | 30.08 | 20.24 | 30.13 |
| 50.8° C. | 20.37 | 30.06 | 20.26 | 30.06 |
| 52.6° C. | 20.40 | 30.21 | 20.24 | 30.07 |
| 55.1° C. | 20.38 | 29.75 | 20.47 | 30.22 |
| 58.2° C. | 20.21 | 29.93 | 19.84 | 29.95 |
| 60.8° C. | 20.44 | 30.16 | 20.23 | 30.03 |
| 62.3° C. | 20.72 | 30.50 | 20.55 | 30.24 |
| 63.0° C. | 21.37 | 30.67 | 20.58 | 30.18 |

Optimal annealing temperature range for VIC probe 1 (shown in Table 2) and YAM probe (shown in Table 2) was determined using two dilutions of viral RNA from respective InfB strains B/Nevada/03/2011 (VIC lineage) and B/Wisconsin/10/2010 (YAM lineage). “High concentration” was represented by 10⁻³ dilution of viral RNA, and “low concentration” was represented by 10⁻⁶ dilution of viral RNA. The tests were performed in triplicate, and average Cₜ values for each annealing temperature were determined. These average Cₜ values are shown in Table 3. Each singleplex assay was conducted using the primers from InfB universal assay described in U.S. Pat. No. 8,241,853, incorporated herein by reference, and either VIC or YAM InfB probes discussed above. All influenza B lineage assays were compared to the CDC Flu rRT-PCR Dx Panel universal InfB assay. The results are shown in “InfB Universal Assay” columns of Table 3. The annealing temperature results shown in Table 3 demonstrated that the VIC and YAM probes tested showed optimal annealing temperatures to be between 50-58° C.

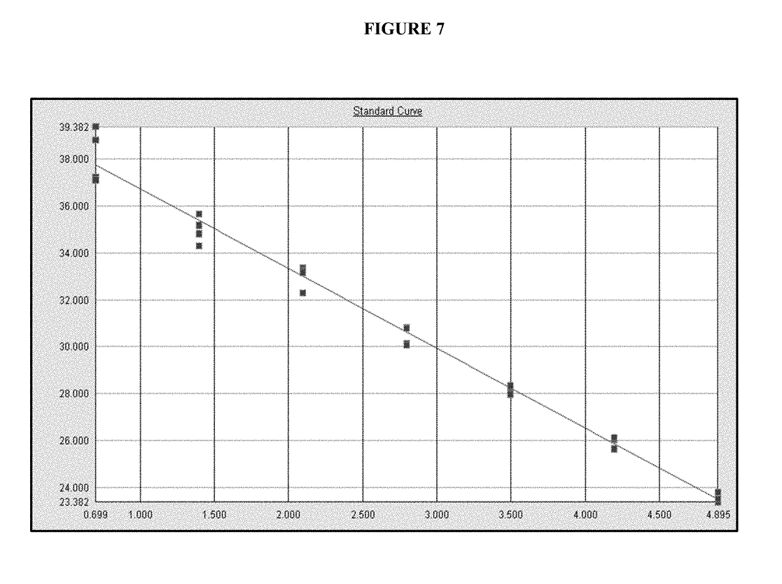

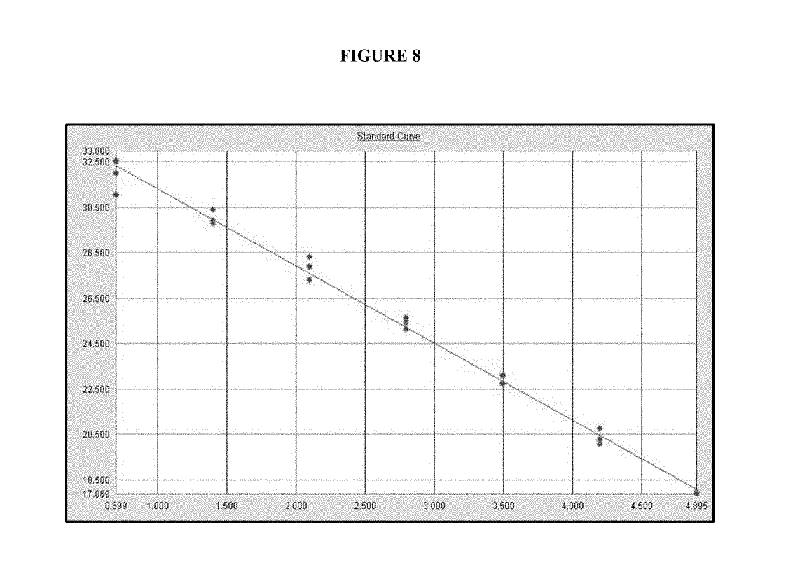

Reaction efficiencies for the YAM and VIC assays using the sets of the primer pairs and the probes discussed above (VIC set 1: VIC forward primer 1, VIC reverse primer and VIC probe 1; YAM set 1: YAM forward primer, YAM reverse primer 1 and YAM probe) were determined by plotting Cₜ values against relative RNA concentrations (RNA dilutions) and by using a linear regression analysis to determine the slope. Absolute quantities of input RNA were not quantified. The slope of the curve as calculated by the Cₜ vs relative RNA concentration (dilution factor) indicated the reaction efficiency. The reaction efficiencies for the YAM and VIC assays performed were estimated to be 96.94 (R2=0.993) and 96.86% (R2=0.988)), respectively. The results of the determination of reaction efficiencies are illustrated in FIGS. 7 and 8, respectively.

### Example 5

### LOD of the Assay

LOD of the assay was determined using quantified viral isolates B/Nevada/03/2011 and B/Texas/06/2011. LOD for each primer and probe set (YAM set 1 and VIC set 1) was confirmed by testing extraction replicates (n=20) of the highest virus dilution where >95% of all replicates tested positive. Virus dilutions were prepared in virus transport medium containing human adenocarcinoma human alveolar basal epithelial cells (A549) cells to emulate clinical specimen matrix. The lowest concentration at which the InfB and VIC or InfB and YAM primer and probe sets tested possessed uniform detection was reported as the LOD value. The LOD value for the VIC assay was determined to be 10^{2.1} EID₅₀/ml. The LOD value for the YAM assay was determined to be 10^{3.5} EID₅₀/ml.

### Example 6

### Sensitivity and Specificity of the Assay

Sensitivity of the assay was evaluated by testing viral RNA isolated from twenty InfB viral isolates (10 Victoria lineage strains, 10 Yamagata lineage strains) from the 2007-2012 influenza seasons with YAM set 1 and VIC set 1 primer and probe sets. The results of the sensitivity testing are summarized in Table 4. Sensitivity assay showed that both VIC and YAM InfB assays detected all influenza viruses from their respective lineages (100% sensitivity).

InfB YAM and VIC assays using YAM set 1 and VIC set 1 primer and probe sets were evaluated for cross-reactivity with the same 20 InfB viruses of the opposite lineage at high titer. No cross-reactivity was detected when tested in triplicate with each isolate of the opposite lineage. Specificity testing was also conducted using grown isolates of influenza A viruses (the results are shown in Table 5) and other non-influenza viral and bacterial respiratory pathogens (the results are shown in Tables 6 and 7) at high infectious titers. False positive results due to cross reactivity were not observed with influenza A, non-influenza viral pathogens, or bacterial respiratory pathogens. Specificity testing revealed 100% specificity of InfB VIC and YAM assays.

### Example 7

### Sensitivity of the Primer and Probe Variants

Sensitivity of the primer and probe variants used InfB YAM and VIC assays was evaluated by testing serial diluted viral RNAs isolated from InfB/Victoria and InfB/Yamagata lineage virus strains that circulated in 2016 and primer and probe variants. The following primer and probe sets were tested: VIC set 1; YAM set 1; VIC set 2: VIC forward primer 2, VIC reverse primer and VIC probe 2; YAM set 2: YAM forward primer, YAM reverse primer 2 and YAM probe (primers and probes are shown in Table 2). The results of the evaluation are summarized in Table 8, A and B. The results from this evaluation demonstrated assay sensitivity for the tested InfB strains was improved by several changes in primer and/or probe sequences.

All patents, patent applications, publications, and abstracts cited above are incorporated herein by reference in their entirety. Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention as defined in the following claims.

TABLE 4
Sensitivity testing

Positive result/# of repetitions

| | | | InfB | |
|---|---|---|---|---|
| | | | Universal | |
| InfB Lineage | Strain Designation | EID₅₀/mL | Assay | VIC |
| InfB/VIC | B/Bolivia/1526/2010 | 10^{1.4} | 3/3 | 3/3 |
| strains | B/Brisbane/33/2008 | 10^{2.4} | 3/3 | 3/3 |
| | B/Brisbane/60/2008 | 10^{1.5} | 3/3 | 3/3 |
| | B/Fujian Gulou/1272/2008 | 10^{2.9} | 3/3 | 3/3 |
| | B/Georgia/07/2010 | 10^{3.2} | 3/3 | 3/3 |
| | B/Hong Kong/230/2009 | 10^{1.2} | 3/3 | 3/3 |
| | B/Hong Kong/259/2010 | 10^{1.2} | 3/3 | 3/3 |
| | B/New Jersey/1/2012 | 10^{1.9} | 3/3 | 3/3 |
| | B/Nevada/03/2011 | 10^{1.2} | 3/3 | 3/3 |
| | B/Texas/26/2008 | 10^{1.2} | 3/3 | 3/3 |

| | | | Positive result/# of repetitions | |
|---|---|---|---|---|
| | | | InfB | |
| | | | Universal | |
| InfB Lineage | Strain Designation | EID₅₀/mL | Assay | YAM |
| InfB/YAM | B/Wisconsin/1/2010 | 10^{2.2} | 3/3 | 3/3 |
| strains | B/Bangladesh/5972/2007 | 10^{1.1} | 3/3 | 3/3 |
| | B/Bangladesh/7110/2007 | 10^{1.6} | 3/3 | 3/3 |
| | B/Chongqingyongchuan/18/2007 | 10^{1.3} | 3/3 | 3/3 |
| | B/Finland/39/2010 | 10^{1.9} | 3/3 | 3/3 |
| | B/Brisbane/3/2007 | 10^{1.4} | 3/3 | 3/3 |
| | B/Hubei-Wujiagang/158/2009 | 10^{1.2} | 3/3 | 3/3 |
| | B/Pennsylvania/7/2007 | 10^{2.2} | 3/3 | 3/3 |
| | B/Santiago/4364/2007 | 10^{2.2} | 3/3 | 3/3 |
| | B/Texas/06/2011 | 10^{1.2} | 3/3 | 3/3 |

TABLE 5
Specificity testing using influenza A virus strains

| | | | Result | | |
|---|---|---|---|---|---|
| | | | InfB | | |
| | | | Universal | VIC | YAM |
| Influenza A Virus | Subtype | EID₅₀/mL | Assay | Assay | Assay |
| A/Brisbane/59/2007 | H1N1 | 10^{8.4} | — | — | — |
| A/California/07/2009 | (H1N1)pdm09 | 10^{8.4} | — | — | — |
| A/Perth/16/2009 | H3N2 | 10^{8.2} | — | — | — |
| A/Minnesota/19/2011 | H1N2v | 10^{7.1} | — | — | — |
| | | (TCID₅₀) | | | |
| A/Indiana/10/2011 | H3N2v | 10^{10.2} | — | — | — |
| A/chicken/Vietnam/NCVD- | H5N1 | 10^{9.1} | — | — | — |
| 016/2008 | | | | | |
| A/Egypt/NO3072/2010 | H5N1 | 10^{9.5} | — | — | — |
| A/Bangladesh/0994/2011 | H9N2 | 10^{10.5} | — | — | — |

TABLE 6
Specificity testing using respiratory pathogens

| | | | | Result | | |
|---|---|---|---|---|---|---|
| Organism | | | | InfB | | |
| | | log TCID₅₀/mL, unless | | Universal | VIC | YAM |
| Virus | Strain | otherwise specified | | Assay | Assay | Assay |
| Enterovirus | Echo 6 | 10^{6.9} | | — | — | — |
| Human Adenovirus, type 1 | Ad.71 | 10^{9.2} | | — | — | — |
| Human Adenovirus, type 7a | S-1058 | 10^{7.1} | | — | — | — |
| Human Coronavirus virus¹ | OC43 | 50.4 | ng/μL | — | — | — |
| Human Coronavirus virus¹ | 299E | 31.6 | ng/μL | — | — | — |
| Human Rhinovirus A | 1A | 10^{5.8} | | — | — | — |
| Human Parainfluenza 1 virus² | NA | 3.0 | ng/μL | — | — | — |
| Human Parainfluenza 2 virus | Greer | 10^{3.1} | | — | — | — |
| Human Parainfluenza 3 virus | C-243 | 10^{7.9} | | — | — | — |
| Respiratory Syncytial virus | CH93-18b | 10^{6.8} | | — | — | — |
| Herpes Simplex Virus | KOS | 10^{8.4} | | — | — | — |
| Varicella-zoster Virus | AV92-3 | 10^{4.4} | | — | — | — |
| Epstein Barr Virus¹ | B95-8 | 1.7 | ng/μL | — | — | — |

| | | | | | |
|---|---|---|---|---|---|
| Measles Virus | Edmonston | 10^{5.2} | — | — | — |
| Mumps Virus | Enders | 10^{7.2} | — | — | — |
| Cytomegalovirus | AD-169 | 10^{6.9} | — | — | — |

¹Organism genomic nucleic acid quantified by spectrophotometry (ng/μL)

TABLE 7
Specificity testing using respiratory pathogens

| Organism | | | | | | |
|---|---|---|---|---|---|---|
| | | Cfu/mL, | | | | |
| | | unless | | Result | | |
| | | otherwise | | InfB | VIC | YAM |
| Bacteria and Yeast | Strain | specified | | assay | assay | assay |
| Bordetella pertussis | A639 | 10^{8.3} | | — | — | — |
| Candida albicans | 2001-21-196 | 10^{8.8} | | — | — | — |
| Chlamydia | TW183 | 40 | IFU/mL | — | — | — |
| pneumoniae¹ | | | | | | |
| Corynebacterium | NA | 10¹⁰ | | — | — | — |
| diphtheriae | | | | | | |
| Escherichia coli | K12 | 10^{9.6} | | — | — | — |
| Haemophilus influenza | M15709 | 10^{6.4} | | — | — | — |
| Lactobacillus | NA | 10^{8.8} | | — | — | — |
| plantarum | | | | | | |
| Legionella | NA | 10^{7.1} | | — | — | — |
| pneumophila | | | | | | |
| Moraxella catarrhalis | M15757 | 10^{9.5} | | — | — | — |
| Mycobacterium | H37Rv | 95 | ng/μL | — | — | — |
| tuberculosis² | | | | | | |

| | | | | | |
|---|---|---|---|---|---|
| Mycoplasma | MI-29 | 10^{7.7} | — | — | — |
| pneumonia | | | | | |
| Neisseria elongate | NA | 10^{8.6} | — | — | — |
| Neisseria meningitides | M2578 | 10^{7.9} | — | — | — |
| Pseudomonas | NA | 10^{10.5} | — | — | — |
| aeruginosa | | | | | |
| Staphylococcus | NA | 10^{10.5} | — | — | — |
| epidermidis | | | | | |
| Staphylococcus aureus | NA | 10^{10.7} | — | — | — |
| Streptococcus | 249-06 | 10^{6.6} | — | — | — |
| pneumoniae | (Thailand) | | | | |
| Streptococcus | 7790-06 | 10^{7.5} | — | — | — |
| pyogenes | | | | | |
| Streptococcus | SS1672 | 10^{8.4} | — | — | — |
| salivarius | | | | | |

¹Organism quantified by Infectious Forming Units (IFU)
²Organism genomic nucleic acid quantified by spectrophotometry (ng/μL)

TABLE 8
Sensitivity of the primer and probe variants
A. InfB/VIC lineage virus strains

| | rRT-PCR Results (Cₜ value) | | |
|---|---|---|---|
| | InfB Universal | VIC assay | |
| InfB Strain and Titer | Assay | VIC set 1 | VIC set 2 |

B/Brisbane/60/2008*

| | | | | | | |
|---|---|---|---|---|---|---|
| 10 ^{5.9} | 25.18 | 26.01 | 27.63 | 29.00 | 25.52 | 25.04 |
| 10 ^{3.9} | 31.81 | 31.67 | 35.31 | 36.14 | 32.40 | 32.39 |

B/Florida/103/2016^{#}

| | | | | | | |
|---|---|---|---|---|---|---|
| 10 ^{4.3} | 24.81 | 25.31 | 32.18 | 31.45 | 25.58 | 25.79 |
| 10 ^{2.3} | 35.87 | 39.73 | — | — | 36.01 | 35.76 |

B/Maryland/15/2016*

| | | | | | | |
|---|---|---|---|---|---|---|
| 10 ^{5.5} | 22.12 | 22.67 | 21.38 | 21.70 | 22.21 | 23.09 |
| 10 ^{3.5} | 31.72 | 32.39 | 31.79 | 31.86 | 31.61 | 31.81 |

*EID₅₀/mL; ^{#}TCID₅₀/mL
B. InfB/YAM lineage virus strains

| | rRT-PCR Results (Cₜ value) | | |
|---|---|---|---|
| | InfB Universal | YAM assay | |
| InfB Strain and Titer* | Assay | YAM set 1 | YAM set 2 |

B/Texas/81/2016

| | | | | | | |
|---|---|---|---|---|---|---|
| 10 ^{5.3} | 19.99 | 19.99 | 22.07 | 21.69 | 20.76 | 20.83 |
| 10 ^{3.3} | 28.80 | 27.99 | 29.99 | 29.99 | 29.80 | 29.26 |

B/Phuket/3073/2012

| | | | | | | |
|---|---|---|---|---|---|---|
| 10 ^{5.9} | 25.21 | 24.83 | 24.31 | 25.91 | 24.96 | 30.79 |
| 10 ^{3.9} | 31.55 | 30.84 | 33.48 | 32.44 | 32.01 | 31.29 |

B/Massachusetts/2/2012

| | | | | | | |
|---|---|---|---|---|---|---|
| 10 ^{5.2} | 23.08 | 22.86 | 23.66 | 23.25 | 24.18 | 24.10 |
| 10 ^{4.2} | 25.59 | 25.56 | 26.59 | 26.33 | 26.47 | 26.69 |

*EID₅₀/mL

1. A probe for or detecting a nucleic acid sequence of a region of hemagglutinin (HA) gene segment of influenza B virus, the probe comprising an oligonucleotide linked to at least one detectable moiety and comprising a sequence at least 90% identical to a sequence selected from the group consisting of SEQ ID NO:3 and SEQ ID NO:6.

2. The probe of claim 1, wherein the sequence is selected from the group consisting of SEQ ID NO:3, SEQ ID NO:6 and SEQ ID NO:8.

3. The probe of claim 1, wherein the oligonucleotide is linked to a fluorophore moiety and a quencher moiety.

4. A kit for performing a real time reverse transcriptase (rRT-PCR) assay, comprising at least one probe of claim 1 and other reagents for conducting the rRT-PCR assay.

5. The kit of claim 4, wherein the kit comprises
a probe comprising the sequence at least 90% identical to SEQ ID NO:3 and
at least one primer selected from the group consisting of
a first primer comprising a sequence at least 90% identical to SEQ ID NO:1
and a second primer comprising a sequence at least 90% identical to SEQ ID NO:2.

6. The kit of claim 5, wherein the kit comprises
a probe comprising the sequence at least 90% identical to SEQ ID NO:6 and
at least one primer selected from the group consisting of
a first primer comprising a sequence at least 90% identical to a sequence SEQ ID NO:4
and a second primer comprising a sequence at least 90% identical to a sequence SEQ ID NO:5.

7. A kit for amplifying and optionally detecting nucleic acid sequence of a region of hemagglutinin (HA) gene segment of influenza B virus in a sample, comprising at least one primer comprising an oligonucleotide having a sequence at least 90% identical to a sequence selected from the group consisting of SEQ ID NO:1, SEQ ID NO:2, SEQ ID NO:4 and SEQ ID NO:5, and other reagents for performing a polymerase chain reaction (PCR).

8. The kit of claim 7, wherein the sequence is selected from the group consisting of SEQ ID NO:1, SEQ ID NO:2, SEQ ID NO:4, SEQ ID NO:5, SEQ ID NO:7 and SEQ ID NO:8.

9. The kit of claim 7, comprising at least one of:
one or both primers for amplifying a region of HA gene of Victoria lineage InfB virus strain selected from the group consisting of a first primer comprising an oligonucleotide of a sequence at least 90% identical to SEQ ID NO:1 and a second primer comprising an oligonucleotide of a sequence at least 90% identical to SEQ ID NO:2,
one or both primers for amplifying a region of HA gene of Yamagata lineage InfB virus strain selected from the group consisting of a third primer comprising an oligonucleotide of a sequence at least 90% identical to SEQ ID NO:4 and a fourth primer comprising an oligonucleotide of a sequence at least 90% identical to SEQ ID NO:5.

10. The kit of claim 9, wherein the one or more other reagents are reagents for performing a real time reverse transcriptase (rRT-PCR) assay for amplifying and detecting the nucleic acid sequence of the region of hemagglutinin (HA) gene segment of influenza B virus.

11. The kit of claim 10, wherein the reagents for performing the rRT-PCR assay comprise at least one of
a first probe for amplification product detection, comprising an oligonucleotide comprising a sequence at least 90% identical to SEQ ID NO:3, if the one or both primers for amplifying a region of HA gene of Victoria lineage InfB virus strain are present in the kit, or
a second probe for amplification product detection, comprising an oligonucleotide comprising a sequence at least 90% identical SEQ ID NO:6, if one or both primers for amplifying a region of PA gene of Yamagata lineage InfB virus strain are present in the kit.

12. A method for amplifying the nucleic acid sequence of the region of hemagglutinin (HA) gene segment of influenza B virus in the sample, comprising performing the PCR using the kit of claim 7.

13. A method of detecting the nucleic acid comprising the region of HA segment of InfB virus in the sample, comprising, performing the rRT-PCR assay using the kit of claim 11.

14. A method of detecting a presence or absence of an InfB influenza virus strain in a sample, comprising:
contacting the sample with reagents for performing a real time reverse transcriptase (rRT-PCR) assay, the reagents comprising a primer and probe set selected from the group consisting of a YAM set and a VIC set,
wherein the YAM set is a probe specific for the region of HA gene of Yamagata lineage InfB virus (YAM probe) and forward and reverse primers specific for the region of HA gene of Yamagata lineage InfB virus (YAM primers), wherein the YAM probe comprises an oligonucleotide comprising a sequence at least 90% identical to SEQ ID NO:6,
and wherein the VIC set is a probe specific for the region of HA gene of Victoria lineage InfB virus (VIC probe) and forward and reverse primers specific for the region of HA gene of Victoria lineage InfB virus (VIC primers), wherein the VIC probe comprises an oligonucleotide comprising a sequence at least 90% identical to SEQ ID NO:3; and,
performing the rRT-PCR assay on the sample to generate a PCR cycle threshold, wherein if the cycle threshold is below a control value, the InfB virus strain is absent from the sample, and wherein if the cycle threshold is above the control value, the InfB virus strain is present in the sample.

15. The method of claim 14, wherein the YAM primers comprise one or both primers selected from the group consisting of a forward primer comprising a sequence at least 90% identical to SEQ ID NO:4 and a reverse primer comprising sequence at least 90% identical to SEQ ID NO:5.

16. The method of claim 15, wherein the forward primer comprises SEQ ID NO:4 and the reverse primer comprises SEQ ID NO:5 or SEQ ID NO:9, and the YAM probe comprises SEQ ID NO:6.

17. The method of claim 14, wherein the VIC primers comprise one or both primers selected from the group consisting of a forward primer comprising a sequence at least 90% identical to SEQ ID NO:1 and a reverse primer comprising sequence at least 90% identical to SEQ ID NO:2.

18. The method of claim 17, wherein the forward primer comprises SEQ ID NO:1 or SEQ ID NO:7, the reverse primer comprises SEQ ID NO:2, and the VIC probe comprises SEQ ID NO:3 or SEQ ID NO:8.

19. The method of claim 14, wherein the sample is a sample derived from a human or an animal subject, a laboratory sample, a virus isolate sample or a vaccine sample.

20. A method of determining if a subject is infected with an InfB virus strain, comprising performing the method of claim 14 on a sample derived the subject, wherein the subject is not infected if the InfB virus strain is absent from the sample, or wherein the subject infected if the InfB virus strain is present in the sample.


SEQUENCE LISTING (9 sequences)

SEQ ID NO: 1 - DNA, 20 bp, Artificial sequence
  Synthetic construct
gatctggacgtagccttggg

SEQ ID NO: 2 - DNA, 22 bp, Artificial sequence
  synthetic construct
taacaggtctgacttcatggag

SEQ ID NO: 3 - DNA, 16 bp, Artificial sequence
  synthetic construct
ttccccgtgcattttg

SEQ ID NO: 4 - DNA, 20 bp, Artificial sequence
  Synthetic construct
gatctggatgtggccttggg

SEQ ID NO: 5 - DNA, 20 bp, Artificial sequence
  Synthetic construct
  Y (10)..(10): C or T
aggtctgacytcgtgragta

SEQ ID NO: 6 - DNA, 15 bp, Artificial sequence
  Synthetic construct
cacacacattggcct

SEQ ID NO: 7 - DNA, 20 bp, Artificial sequence
  Synthetic construct
gatctggatgtagccttggg

SEQ ID NO: 8 - DNA, 16 bp, Artificial sequence
  Synthetic construct
ttccccgtgcattttg

SEQ ID NO: 9 - DNA, 23 bp, Artificial sequence
  Synthetic construct
  Y (12)..(12): C or T
acaggtctgacytcatggagtat